(19) United States
(12) Patent Application Publication
(10) Pub. No.: US 2018/0282786 A1
(43) Pub. Date: Oct. 4, 2018

(54) METHODS AND APPARATUS FOR SELECTIVE NUCLEIC ACID SEPARATION

(71) Applicants: Michael Joseph Pugia, Ganger, IN (US); Zane Baird, Brigham City, UT (US); Zehui Cao, Carmel, IN (US)

(72) Inventors: Michael Joseph Pugia, Ganger, IN (US); Zane Baird, Brigham City, UT (US); Zehui Cao, Carmel, IN (US)

(21) Appl. No.: 15/941,125

(22) Filed: Mar. 30, 2018

Related U.S. Application Data
(60) Provisional application No. 62/480,367, filed on Apr. 1, 2017.

(51) Int. Cl. C12Q 1/682; C12N 15/10

(52) U.S. Cl. CPC C12Q 1/682; C12N 15/1096; C12Q 2563/107; C12Q 2561/113; C12Q 2561/101; C12Q 2563/131

(57) ABSTRACT

Methods are provided for the selective isolation, amplification and detection of nucleic acids from samples, said method comprising: (a) enriching selectively said nucleic acids present in said samples on a binding matrix; (b) releasing said nucleic acids from the binding matrix; (c) selectively amplifying said nucleic acids; and (d) analysing said amplified nucleic acids.

This application claims the priority benefit under 35 U.S.C. section 119 of U.S. Provisional Patent Application No. 62/480,367 entitled “Methods And Apparatus For Selective Nucleic Acid Separation” filed on Apr. 1, 2017; and which is in its entirety herein incorporated by reference.

### BACKGROUND OF THE INVENTION

The instant invention relates to methods for selective the enrichment and analysis of rare nucleic acids in the presence of non-rare nucleic acids. In some aspects, the invention relates to methods, apparatus and kits for selectively enriching, amplifying and detecting one or more different populations of rare nucleic acids in a sample suspected of containing one or more different populations of rare nucleic acids and non-rare nucleic acids. In some aspects, the invention relates to methods and kits for detecting one or more different populations of rare nucleic acids that are freely circulating in samples. In some aspects, the invention relates to methods and kits for detecting one or more different populations of rare nucleic acids that are associated with rare cells in a sample suspected of containing one or more different populations of rare cells and non-rare cells.

The detection of rare molecules can be achieved by conventional nucleic acid assays. However, the nucleic acids must be subjected to one or more lengthy purification steps and amplifications that can take several days for analysis time. For example, amplification techniques include, but are not limited to, enzymatic amplification such as, for example, polymerase chain reaction (PCR), ligase chain reaction (LCR), nucleic acid sequence based amplification (NASBA), Q-β-replicase amplification, 3SR (specific for RNA and similar to NASBA except that the RNAase-H activity is present in the reverse transcriptase), transcription mediated amplification (TMA) (similar to NASBA in utilizing two enzymes in a self-sustained sequence replication), whole genome amplification (WGA) with or without a secondary amplification such as, e.g., PCR, multiple displacement amplification (MDA) with or without a secondary amplification such as, e.g., PCR, whole transcriptome amplification (WTA) with or without a secondary amplification such as, e.g., PCR or reverse transcriptase PCR, for example.

The detection of rare molecules in the range of single copies (attomolar 10⁻¹⁸ M nucleotides per μL) cannot be achieved by conventional nucleic acid assays, which require a number of molecular copies far above the numbers found for rare molecules. Most nucleic acid methods require nanomolar (10⁻⁹ M) quantities for detection of nucleotides. An amplification of ˜10⁹ is often required to generate enough copies for detection. However, amplification errors tend to propagate in the amplified materials to unacceptable error rates (poor fidelity) when pushed beyond limits. For example, PCR can rather accurately complete 20 cycles for ˜10⁵ copy number amplification of a 300 base pair targets, but if pushed to >30 cycles needed for ˜10⁹ copy number amplification, yields a 20% error rate when using a polymerase with fidelity of 2×10⁻⁵ mutations/bp/template doubling.

The detection of rare nucleic acids that are circulating in the sample are typically a mixture of rare and non-rare nucleic acids. The materials can be cellular, e.g. internal to cells or “cell free” material and not bound or associated to any intact cell. Cell free nucleic acids are important in medical applications such as, for example, diagnosis of many specific tumor mutations in tissues are detected by circulating cell free DNA (cfDNA). It is understood that cfDNA correlates to the total amount of tumor distributed throughout the body, and is therefore a measure of tumor burden. Cell free analysis requires isolation and detection of nucleic acids from a very small fraction of nucleic acids in sample. When cell free nucleic acids are shed into the peripheral blood from diseased cells in tissues, these nucleic acids are mixed with nucleic acids from normal cells. For example, approximately 10⁹ cells are present in a cubic cm of diseased tissue. If this entire tumor was dissolved into the 5 L of blood in the body this would be 2 million cells per 10 mL blood tube. The actual tumor size to allow such dissolved material is of course greater. The 2 million cells per blood tube give a lot of genomic DNA at 3 million bps as 300 nucleated cells contain about 1 ng of genomic DNA. However cfDNA is typically a fragment of 85 to 230 bp, meaning there is only 0.4 ng of cfDNA/blood tube. The observed reference range for normal cfDNA in blood is between 0.36 to 50.5 μg/blood tube. Therefore purity of rare cell free nucleic acid is extremely low at only 0.01% or less even for large tumor masses.

The detection of rare nucleic acids that are cell bound or included in a cell is also important in medical applications such as, for example, diagnosis of many diseases that can be propagate from a single cell. The analysis of nucleic acids of certain rare cells has extremely important medical applications, and requires isolation and detection of nucleic acids from very small fraction of cells in sample under analysis. For example, circulating tumor cells (“CTCs”) are of particular interest in the diagnosis of metastatic cancers. In conventional methods, CTC are isolated from a 10 mL whole blood sample by first removing red blood cells (RBCs) by lyses and leaving a few hundred CTCs mixed with about 800,000,000 white blood cells (“WBCs”). In second step, the sample can be filtered, to a few CTCs mixed with about 15,000 WBC. Therefore, purity of rare cell is extremely low and only 0.01% to 0.00001% even after enrichment steps.

The problem of purity is further complicated as the cell has many types of nucleic acids. For example, while each cell has 10 to 30 pg of total RNA, only 1-5% of this is mRNA (360,000 copies of 12,000 different mRNA types), while 80-85% is rRNA and 10-15% is low molecular weight RNA (tRNA and snRNA). Additional, while each cell has 6 pg of total DNA, this represents 3.2 billion base pairs and 70,000 genes. Thus the impurity can be much greater in a sample depending on the type of nucleic acid and the gene desired to be measured.

Low purity causes problems in the amplifications as samples do not contain the minimum amount of desired nucleic acid needed for analysis, typically between 10 ng to 3.0 μg per sample. Also, low purity sample introduces more inhibitors and can favor non-specific nucleic acid amplification due to more ideal fragment size and melting temperatures. This loss of efficiency further reduces the amplified contraction and propagates errors.

Therefore, methods with high separation and washing efficiency of rare nucleic acids are particularly important. The current state of the art for rare nucleic acid purification has several issues, which keep rare cell molecular analysis from being competitive with routine systems. Many of the current approaches to purify cell free rare nucleic acids are non-specific and isolate all nucleic acids. These include separation methods like precipitation evaporation, membrane filtration, extraction with organic solvents, centrifugation methods such as differential, zonal, lysis, isopycnic and others, electrophoresis, chromatography such as ionic, affinity, gel and other, adsorption onto silica using a chaotropic salt, for binding and release e.g. membranes, spin columns and magnetic nanoparticles. For example in U.S. Pat. No. 8,703,931, all nucleic acids are captured on silica coated magnetic beads. The beads are separated by a magnetic field and washed to remove proteins, nucleases, and other cellular impurities. The nucleic acids are eluted in a small volume of elution buffer for subsequent analysis. However even after this method, the nucleic acids remain extremely impure, only 0.01% pure. Additionally, the low affinity of these approaches causes incomplete removal of rare nucleic acid and samples with few copies of rare nucleic acid (<10⁴) are missed.

Another current approach to purify cell free rare nucleic acids, is to use nucleic acid affinity agents that are specific and isolate more of the rare nucleic acids and less of the non-rare nucleic acids. A hybridization oligo is a widely used nucleic acid affinity agent. In U.S. Pat. No. 5,512,439 affinity purification by hybridization is carried out on magnetic beads. The beads are separated by a magnetic field and washed to contain only the nucleic acid hybridized to the probe (affinity agent). The nucleic acids are eluted in a small volume of elution buffer for subsequent analysis. However, this method is not selective for cells and would extract nucleic acids from non-rare cells. A key problem with this approach is hybridization reactions often fail when sample is extremely impure, e.g. lower than 0.01%, as non-rare nucleic acid prevent binding to probe. The issue is that the affinity of the nucleic acid affinity agent is not strong enough to selectively bind and remove a rare nucleic acid in the presence of large excess of non-rare nucleic acids. Incomplete removal of rare nucleic acid occurs and samples with few copy (<10⁴) are missed.

Several new approaches for selective removal of rare nucleic acids use proteins that bind RNA (Jazurek Nucleic Acids Research, 2016). In doing so, these approaches also remove the RNA. In one approach, the RNA is tagged in vivo or in vitro synthesis with an affinity label like biotin by incorporating specifically modified ribonucleoside tri-phosphate (rNTPs) during RNA synthesis. Other approaches use nucleic acid affinity agents such as RNA or DNA binding proteins, antibodies or aptamers. For example, an aptamer is a nucleic acid structure that can be incorporated into the RNA and bound to a protein selectively, such as the MS2-binding RNA stem-loop binding interaction. However, these approaches require in vivo or in vitro synthesis which requires living cells to be regenerated in costly and time-consuming methods. Additionally, these approaches require a means to remove the nucleic acid affinity agents. Cross linking can be used to remove the nucleic acid affinity agents. For example, cross linking to UV reactive groups, or by formation of Schiff bases from aldehyde, and formaldehyde reactive groups. These groups can be included on peptides, proteins and nucleic acids. In another approach, a modified nuclease-inactive Cas9 protein (dCas9), an associated guide RNA that matches the target RNA sequence, and a short protospacer adjacent motif (PAM) are used to capture target DNA. A PAM is a 2-6 base pair DNA sequence immediately following the DNA sequence targeted by the Cas9. While these methods have been successfully used to provide selective binding of nucleic acids, the use of the eluted nucleic acids for subsequent molecular analysis is not often possible as the binding proteins do not release the nucleic acids sufficiently for amplification.

The problems with all methods are further complicated as some nucleic acids can be unstable and fragmented. For example, prokaryotic mRNA only has a 2 min half-life and eukaryotic mRNA has a 30 min to 5 h half-life. While DNA is relatively stable, the action of enzyme and other chemicals in the sample can alter the DNA. Integrity problems include degradation, fragmentation, and binding and crosslinking of nucleic acids. The nucleic acid size, structure and sources greatly influence stability. Fixation is often used to stabilize samples. However, fixation causes problems as nucleic acids are usually heavily fragmented and chemically modified by a fixation agent such as, for example, formaldehyde. Although formaldehyde modification cannot be detected in standard quality control assays such as, for example, gel electrophoresis, it does strongly interfere with analysis of nucleic acids. While nucleic acid isolation and purification methods can be optimized to reverse as much formaldehyde modification as possible without further RNA degradation, RNA purified from fixed samples are not a good candidate for downstream applications that require full-length RNA such as, for example, polymerase chain reaction methods.

The problem of purity and stability of nucleic acids is further exacerbated by the chemicals used in these methods of isolation. Such methods employ reagents such as, for example, detergents, solvents or phenols, which can damage the nucleic acid material. Furthermore, contamination of nucleic acids with other reagents such as organic solvents and other extraction chemicals can affect the integrity of nucleic acid samples. Nucleases and nuclease inhibitor contamination can reduce amplification of isolated nucleic acids.

Filtration is another method used for the separation and washing of nucleic acids, wherein cells with nucleic acids are captured onto particles. Filtration relies on using a porous matrix and as a useful method to sort rare cells by size or nature for pre-enrichment. During filtration, smaller non-rare cells are lost and larger rare cells separated. However, as mentioned above, filtration techniques can only yield low 0.1% purity or less, thus again highly accurate and sensitive detection methods and pre-enrichment are required. Additional filtration methods require means to remove nucleic acid from the porous matrix. Approaches such as laser microdissection, lifting and punching to remove individual cells require too much time and damage the cells nucleic acids. Additionally, individual cells can be unstable and very heterogenic, and yield poor quality or non-representative nucleic acids. Solutions to the above problems are presented in PCT/US2015/038990, where buffers are used to allow release of nucleic acid from non-rare cells. However purity is not improved beyond an order of magnitude of 0.1%. In U.S. application Ser. No. 14/891,423 sonication is used to remove cells but this is also non-selective and destroys the nucleic acids.

There is, therefore, a long felt need to develop reagent, methods and apparatus that provide for specific or selective isolation of rare nucleic acids and for delivery into a mass spectrometer while avoiding dilution of the detection liquid.

### SUMMARY OF THE INVENTION

Some examples in accordance with the principles described herein are directed to methods for selective enrichment, amplification and analysis of rare nucleic acids in the presences of non-rare nucleic acids. The method allows high purity nucleic acid detection that is resistant to amplification error. The method also allows nucleic acid enrichment, amplification and analysis that is selective to target rare nucleic acid and is resistant to amplification error.

In some aspects, the invention relates to methods, apparatus and kits for selectively enriching, amplifying and detecting one or more different populations of rare nucleic acids in a sample suspected of containing one or more different populations of rare nucleic acids and non-rare nucleic acids. In some aspects, the invention relates to methods and kits for detecting one or more different populations of rare nucleic acids that are freely circulating in samples. In some aspects, the invention relates to methods and kits for detecting one or more different populations of rare nucleic acids that are associated with rare cells in a sample suspected of containing the one or more different populations of rare cells and non-rare cells.

Some examples in accordance with the principles described herein are directed to methods, apparatus and kits where nucleic acid enrichment occurs on a nucleic acid binding matrix, where nucleic acids are released from a nucleic acid binding matrix, released nucleic acids are selectively amplified, and nucleic acid corrected analysis performed.

Some examples in accordance with the principles described herein are directed to methods of selective enrichment where a sample has been separated into a sample containing cellular rare nucleic acid, and is enrichment on nucleic acid binding matrix, where nucleic acids are released from nucleic acid binding matrix, released nucleic acids are selectively amplified, and nucleic acid corrected analysis performed.

Some examples in accordance with the principles described herein are directed to methods of selective enrichment where a sample has been separated into a sample containing cell free nucleic acid, and is enriched on a nucleic acid binding matrix, where nucleic acids are released from a nucleic acid binding matrix, released nucleic acids are selectively amplified, and nucleic acid analysis performed correctly.

Some examples in accordance with the principles described herein are directed to methods of selective enrichment where a sample has been separated into a sample containing cellular rare nucleic acid and a sample containing cell free rare nucleic acids, and may be enriched separately on a nucleic acid binding matrix, where the nucleic acids are released from the nucleic acid binding matrix, released nucleic acids are selectively amplified, and nucleic acid analysis performed correctly.

Some examples in accordance with the principles described herein are directed to methods of selective enrichment where a sample contains cellular rare nucleic acid and cell free rare nucleic acids, and may be enriched together on a nucleic acid binding matrix, where nucleic acids are released from the nucleic acid binding matrix, released nucleic acids are selectively amplified, and nucleic acid analysis performed correctly.

Some examples in accordance with the principles described herein are directed to methods of selective enrichment where a sample contains cellular rare nucleic acid and cell free rare nucleic acids, and cellular rare nucleic acid may be enriched first on a nucleic acid binding matrix, cell free nucleic acids are not enriched and pass through the nucleic acid binding matrix, cell nucleic acids are released from nucleic acid binding matrix first, and both cell and cell free released nucleic acids are selectively amplified separately or combined, and nucleic acid analysis performed correctly.

Some examples in accordance with the principles described herein are directed to methods of selective enrichment where a sample contains cellular rare nucleic acid and cell free rare nucleic acids, and cell free rare nucleic acid may be enriched first on a nucleic acid binding matrix, cell nucleic acids are not enriched and pass through the nucleic acid binding matrix, cell free nucleic acids are released from nucleic acid binding matrix followed by cell nucleic acids, and both cell and cell free released nucleic acids are selectively amplified separately or combined, and nucleic acid analysis correctly performed.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a schematic in cross-section depicting an example of an apparatus, method or kit in accordance with the principles described herein of selective enrichment where sample 1 has been separated into a sample containing cellular rare nucleic acids 2 enriched on nucleic acid binding matrix 3 with some normal cellular nucleic acids 4 remaining and where nucleic acids 5 are released from nucleic acid binding matrix to form a mixture of disease-related nucleic acids 6 and reference nucleic acids 7, and where released cellular rare nucleic acids 8 are selectively amplified such that rare nucleic acids are amplified, and where nucleic acid corrected analysis is performed and determining whether cellular rare nucleic acids 9 are present over cellular normal cell nucleic acid 10.

FIG. 2 is another schematic in cross-section depicting an example of an apparatus, method or kit in accordance with the principles described herein of selective enrichment where sample 1 has been separated into a sample containing cell free disease-related nucleic acids 2 and cell free reference nucleic acids 3 enriched on a nucleic acid binding matrix 4 and where nucleic acids are released 4′ from nucleic acid binding matrix 4 to form a mixture of disease-related nucleic acids 2 and reference nucleic acids 3, and where released cellular rare nucleic acids are selectively amplified 5 such that rare nucleic acids are amplified, and where nucleic acid corrected analysis performed and determining whether cell free rare nucleic acids 6 are present over cellular normal cell free nucleic acid 7.

FIG. 3 is an additional schematic in cross-section depicting an example of an apparatus, method or kit in accordance with the principles described herein of selective enrichment where sample 1 has been separated into a cellular rare nucleic acid 2 enriched on a nucleic acid binding matrix 3 with some normal cellular nucleic acid 4 remaining and sample containing cell free disease-related rare nucleic acids 5 and cell free reference rare nucleic acid 6 enriched on nucleic acid binding matrix 3 and where nucleic acids are released 7 from nucleic acid binding matrix to form a mixture of disease-related nucleic acids 5 and reference nucleic acids 6, and where released cellular rare nucleic acids 5 are selectively amplified 8 such that rare nucleic acid are amplified, and wherein nucleic acid corrected analysis is performed and determining whether cellular or cell free rare nucleic acid 9 are present over cellular or cell free normal cell nucleic acid 10.

### DETAILED DESCRIPTION OF THE INVENTION

### General Discussion

Methods, apparatus and kits in accordance with the principles described herein have application in any situation where rare nucleic acids are required. Examples of such applications include, by way of illustration and not limitation, methods of isolation, amplification, and detection of nucleic acids from a sample selective for rare nucleic acid. Examples in accordance with the principles described herein are directed to nucleic acid analysis.

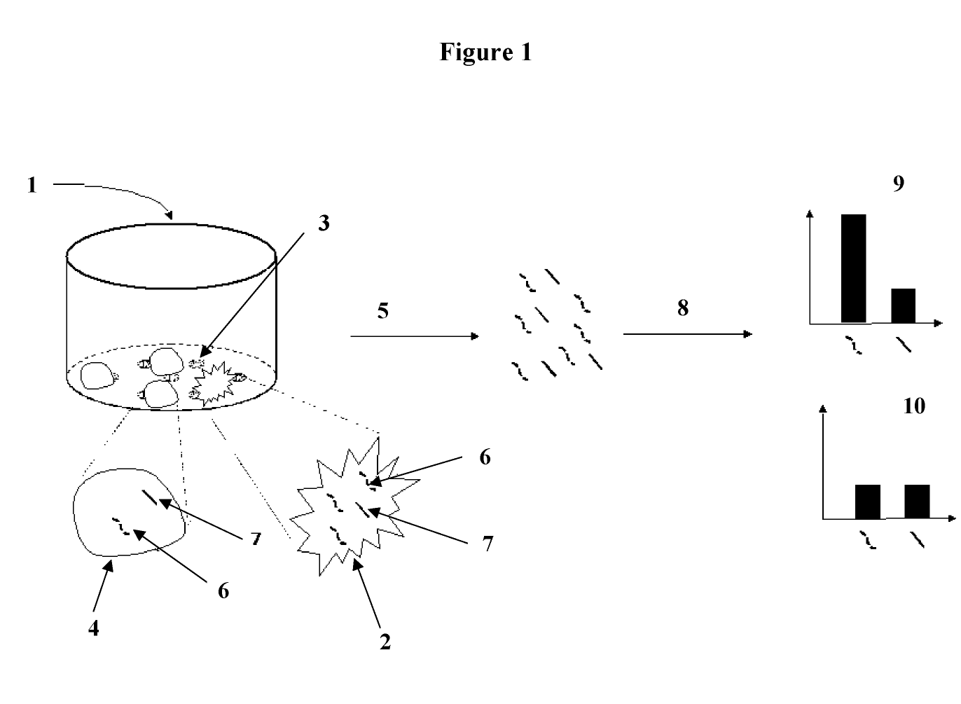

An example of an example of an apparatus, method or kit for isolation of nucleic acids in accordance with the principles described herein is depicted in FIG. 1. FIG. 1 is a schematic depicting an in accordance with the principles described herein of selective enrichment of a cellular rare nucleic acid onto a nucleic acid binding matrix, where the nucleic acids are released from the nucleic acid binding matrix, where released cellular rare nucleic acids are selectively amplified and where nucleic acid corrected is analyzed.

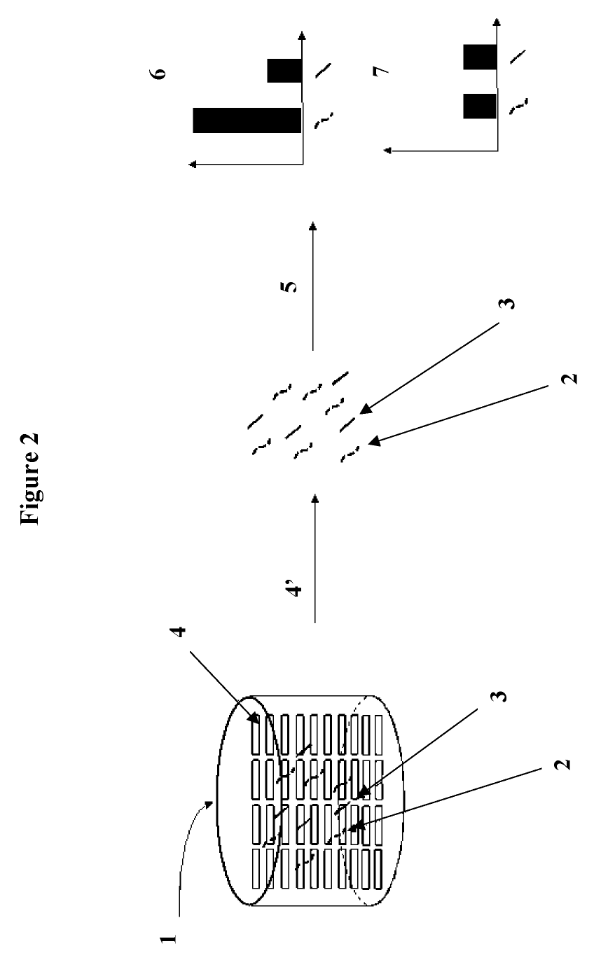

An example of another apparatus, method or kit for isolation of nucleic acids in accordance with the principles described herein is shown in FIG. 2. FIG. 2 is a schematic depicting in accordance with the principles described herein of selective enrichment of cell free rare nucleic acids onto a nucleic acid binding matrix where released cellular rare nucleic acids are selectively amplified and where nucleic acid corrected analysis is analyzed.

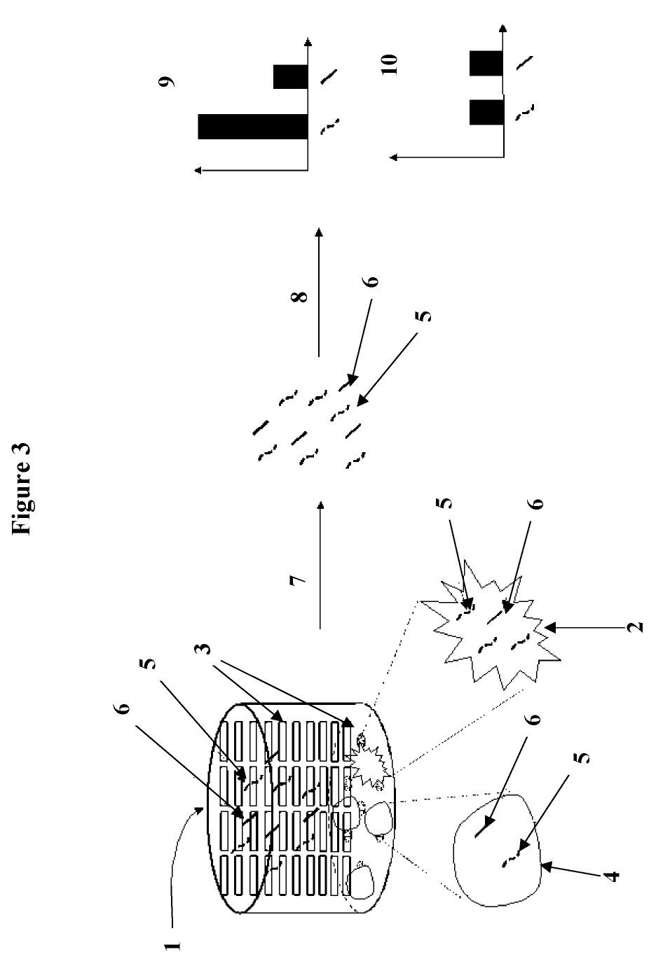

A further example of an apparatus, method or kit for isolation of nucleic acids in accordance with the principles described herein is illustrated in FIG. 3. FIG. 3 is a schematic depicting an in accordance with the principles described herein of selective enrichment a cellular and cell free rare nucleic acid onto a nucleic acid binding matrix, where the nucleic acids are released from nucleic acid binding matrix, where released rare cellular and cell free rare nucleic acids are selectively amplified and where nucleic acid corrected analysis is analyzed.

Some examples in accordance with the principles described herein are directed to methods of selective enrichment, selected amplification and corrected detection of rare nucleic acid such that an enrichment, used a nucleic acid binding affinity agents which includes a porous matrix either alone or with additional nucleic acid affinity agents.

Some examples in accordance with the principles described herein are directed to methods of selective enrichment, selected amplification and corrected detection of rare nucleic acid such that on enrichment, non-rare nucleic acids are removed from the nucleic acid affinity agent by washing solution, and retained rare nucleic acids are released from the nucleic acid affinity agent using a release solution.

Some examples in accordance with the principles described herein are directed to methods of selective enrichment, selected amplification and corrected detection of rare nucleic such that released rare nucleic acids are selectively amplified form a mixture of disease-related nucleic acids and reference nucleic acids and amplified mixture a corrected analysis performed to determine the presence of rare nucleic acids over non-rare nucleic acids.

Some examples in accordance with the principles described herein are directed to methods of selective enrichment, selected amplification and corrected by ratio of disease-related nucleic acids to reference nucleic acids for determining whether rare nucleic acid are present.

Other examples in accordance with the principles described herein are directed to method of selective isolation and amplification of nucleic acid such that, specific nucleic acid released undergoes a “nucleic acid enrichment” to generate a minimum copy number of rare nucleic acids in the presence in a maximum impurity of non-rare nucleic acids and can be further amplified by a minimum cycle such that sample can be split into more than one aliquot, the aliquot can be removed for performing nucleic acid corrected analysis.

The term “nucleic acid binding matrix” refers to a material capable to selectively bind to nucleic acids and includes a “porous matrix”, either alone or with additional “nucleic acid affinity agents”, “capture particle”, “cell affinity agents” or “hybridization oligo” materials in any combination. The term “porous matrix” refers to a matrix that is a solid material, which is impermeable to liquid except through one or more pores of the matrix. The term “capture particle” refers to particles bound to nucleic acid affinity agents, or cell affinity agents and hybridization oligo. The term “nucleic acid affinity agent” refers to a molecule capable of selectively binding to nucleic acids. The term “cell affinity agent” refers to a rare cell markers capable of binding selectively to rare cell. The term “hybridization oligo” refers to a nucleic acid (e.g., polynucleotide) that is complementary to a rare nucleic acid to be detected. The phrase “selective enrichment” means that the nucleic acid binding matrix distinguishes and enriches for one group of nucleic acids from another group of nucleic acids.

The phrase “rare nucleic acids” refers to nucleic acids that may be detected in a sample where the nucleic acids are indicative of population of fewer nucleic acids in population of excess non-rare nucleic acids. The phrase “population of rare nucleic acids” refers to a group of nucleic acids that share a common nucleic acid that is specific for the group of nucleic acids. These “rare nucleic acids” can be “disease-related nucleic acids” and can be “reference nucleic acids”. The term “disease-related nucleic acids” means a nucleic acid that can distinguish an abnormal condition from the normal condition. The term “reference nucleic acids” means a nucleic acid that is present in both rare and non-rare cells at similar level. These “rare nucleic acids” can be “cellular rare nucleic acids” and “cell free rare nucleic acids”. The phrase “cellular rare nucleic acids” refers to rare molecules that are bound in a cell and may or may not freely circulate in a sample. The phrase “cell free rare nucleic acids” refers to rare molecules that are not bound to a cell and/or that freely circulate in a sample.

The term “selective amplification” refers to replication of rare nucleic acid sequences or segments of the sequences to preferentially increase the total copy numbers of these sequences or sequence segments over non-rare nucleic acid sequences. The term “high fidelity amplification” is an amplification of the lowest number of non-rare nucleic acid molecules that contaminate the rare nucleic acids as the result of a low error rate in duplicating the rare nucleic acid molecules. The term “minimal copy number” is the lowest number of rare nucleic acid molecules that can be detected by a method. The term “minimal purity” is the lowest number of rare nucleic acid that can be detected by a method. The term “minimal cycle number is the lowest number of amplification that are needed for detection of rare nucleic acids while a “high fidelity amplification” is maintained.

The term “nucleic acid analysis” refers to using analytical methods to confirm the presence of or identify or quantify the target nucleic acid sequences. The term “selective amplification” refers to preferential amplification of rare nucleic acid over non-rare nucleic acids. The term “nucleic acid corrected analysis” refer to correction of “disease-related nucleic acids” by using “reference nucleic acids” such that “rare nucleic acids” are detected.

The nucleic acid corrected analysis is done using reference materials such as reference nucleic acids as an internal standard of the samples being analyzed. As is well known in the art, the identification of the biological substances may involve one or more comparisons with reference specimens. The reference specimen may be obtained from the same subject or from a different subject who is either not affected with the disease or is a patient. The reference specimen could be obtained from one subject, multiple subjects or be synthetically generated. The identification may also involve the comparison of the identification data with the databases to identify the biological substance.

Internal standard: An appropriate internal standard can be spiked in a well defined concentration in every sample to increase the precision in relative and absolute quantitation. This internal standard deals as a reference and is used to compensate for any technical variations between individual measurements. Typically, such an internal standard is composed of a well known nucleic acid or any other similar molecule with very similar physico-chemical properties than the target molecule. The similarity between internal standard and target molecule is needed to ensure a similar response of both molecules to any technical variation during the measurement.

The term “reference nucleic acid” as used herein refers to a nucleic acid which is intended to be identified for the purposes of comparison with genomic nucleic acid under investigation. Reference nucleic acid may be a DNA or RNA, natural or synthetic. In certain cases, the reference nucleic acid may contain relatively invariant sequence i.e. a housekeeping gene or locus or other gene, or other sequence in a chromosome that is not expected to change under varying conditions (e.g., a normal state or a disease state). A reference nucleic acid may also represent a nucleic acid in a normal or wild type state, that is, absent point mutations, translocations, deletions, or duplications. In another case, a reference nucleic acid may represent a nucleic acid sequence with point mutations, translocations, deletions, or duplications. In some cases, the genomic nucleic acid under investigation and the reference nucleic acid may be obtained from the same sample. In other cases, the genomic nucleic acid and the reference nucleic acid may be obtained from different samples. In some cases, reference nucleic acid may be obtained from a different source than the genomic nucleic acid. In some cases, reference nucleic acid may be obtained from a different organism than the genomic nucleic acid. The method according to the present invention can, subsequent to determination of an existing ratio of a target nucleic acid and an internal standard nucleic acid, determine correctly a concentration or amount of the target nucleic acid based on the obtained existing ratio.

The term “bound” refers to the manner in which two moieties are associated to one another. The association is through non-covalent binding such as ionic binding, hydrophobic binding, pocket binding and the like.

The term “attachment” refers to the manner in which two moieties are bound accomplished by a direct bond between the two moieties or a linking group can be employed between the two moieties.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

### Examples of Selective Enrichment

Selective enrichment increases the concentration of the one or more different populations of rare nucleic acid over that of the non-rare nucleic acid to form a concentrated sample. In some examples, the sample is subjected to a filtration procedure using a porous matrix that retains the rare nucleic acid while allowing the non-rare nucleic acid to pass through the porous matrix thereby enhancing the concentration of the rare nucleic acid. In some examples, one or more rare nucleic acids are non-cellular, and the sample is combined with additional nucleic acid binding matrix entities to bind rare nucleic acid over non-rare nucleic acid to form a concentrated sample. In other examples, such as one or more rare nucleic acid are cellular or associated with a cell, and the sample is combined with additional nucleic acid binding matrix entities to bind rare nucleic acid over non-rare nucleic acid to form a concentrated sample. In some examples, different types of nucleic acids are separated from one another. For example, DNA and RNA may be separated from one another and from other cellular components such as, e.g., proteins, by methods that include, but are not limited to, differential centrifugation, solvent extraction combined with precipitation using salt, magnetic particle separation, and combinations thereof.

The selective enrichment of rare nucleic acids generates a minimum copy number of rare nucleic acids at a minimal purity of rare nucleic acids in the presence of non-rare nucleic acids such that samples can be further amplified by a minimum cycle such that a high fidelity amplification is maintained. The methods described herein involve trace analysis, i.e., minute amounts of material on the order of 100 to about 10,000,000 minimal copy number of rare nucleic acids. In some examples, the minimum copy number is 100 to about 10,000 copies, 1,000 to about 100,000 copies, 10,000 to about 1,000,000 copies or about 100,000 to about 10,000,000 copies. Since this process involves trace analysis at the detection limits of the nucleic acid analyzers, these minute amounts of material can only be detected when detection volumes are extremely low, for example, 0.1 to about 100 μL. In some examples, the detection volume number is 1 to about 100 μL, or 10 to about 100 μL, or 50 to about 100 μL. Since this process requires selectively amplified rare nucleic acids over non-rare nucleic acid, there is a “minimal purity” of rare nucleic acid that can be amplified, for example, greater than 0.01% to about 20%. In some examples, the minimal purity is 0.01% to about 0.1%, or 0.05% to about 0.1%, or 0.1% to about 1%, or 0.1% to about 20%,

The term “nucleic acid binding matrix” refers to a material able to selectively bind to nucleic acids through the use of “porous matrix” either alone or with additional “nucleic acid affinity agents”, “capture particle”, “cell affinity agents” or “hybridization oligos” materials in any combination. The term “porous matrix” refers to a solid, material, which is impermeable to liquid except through one or more pores of the matrix. The term “capture particle” refers to particles bound to nucleic acid affinity agents, or cell affinity agents and hybridization oligos. The term “nucleic acid affinity agent” refers to a molecule capable of selectively binding to nucleic acids. The term “cell affinity agent” refers to rare cell markers capable of binding selectively to rare cell. The term “hybridization oligo” refers to a nucleic acid (e.g., polynucleotide) that is complementary to a rare nucleic acid to be detected. The phrase “selective” means that the nucleic acid binding matrix distinguishes and enriches for one group of nucleic acids from another group of nucleic acids.

The selective enrichment of rare nucleic acids removes non-rare nucleic acids from the nucleic acid binding matrix by washing with solution. The washing is conducted with a solution containing solvents, chemicals, surfactants, salts, polymers or other material and reagents typically used for nucleic acid analysis. After removing non-rare nucleic acids from the nucleic acid binding matrix, selective enrichment removes rare nucleic acids from the nucleic acid binding matrix by eluting with a solution. The elution is conducted with a solution containing solvents, chemicals, surfactants, salts, polymers or other material and reagents typically used for nucleic acid analysis.

The combination of the sample and the nucleic acid binding matrix is held for a period of time and at a temperature to permit the binding of rare nucleic acids with the nucleic acid binding matrix, a hydrodynamic force such as a vacuum is applied to the sample on the porous matrix to facilitate passage of non-rare nucleic acids, non-rare cells and other particles through the matrix. The level of vacuum applied is dependent on one or more of the nature and size of the different populations of rare cells, non rare cells, nucleic acid binding matrix, nucleic acids, reagents, the nature of the porous matrix, and the size of the pores of the porous matrix.

Contact of the sample with the nucleic acid binding matrix is continued for a period of time sufficient to achieve retention of rare nucleic acids and/or rare cells on a surface of the porous matrix to obtain a surface of the porous matrix having an enriched populations of rare nucleic acids and/or rare cells as discussed above. The period of time is dependent on one or more of the nature and size of the different populations of rare nucleic acids and/or rare cells rare molecules, the nature of the porous matrix, the size of the pores of the porous matrix, the level of vacuum applied to the blood sample on the porous matrix, the volume to be filtered, and the surface area of the porous matrix. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes.

An amount of each different nucleic acid binding matrix that is employed in the methods in accordance with the principles described herein is dependent on one or more of the nature and potential amount of each different population of rare nucleic acids or rare cells, the nature of the nucleic acid binding matrix, the nature of the cells if present, the nature of a particle if employed, and the amount and nature of a blocking agent if employed. In some examples, the amount of each different nucleic acid binding matrix employed is about 0.001 μg/μL to about 100 μg/μL, or about 0.001 μg/μL to about 80 μg/μL, or about 0.001 μg/μL to about 60 μg/μL, or about 0.001 μg/μL to about 40 μg/μL, or about 0.001 μg/μL to about 20 μg/μL, or about 0.001 μg/μL to about 10 μg/μL, or about 0.5 μg/μL to about 100 μg/μL, or about 0.5 μg/μL to about 80 μg/μL, or about 0.5 μg/μL to about 60 μg/μL, or about 0.5 μg/μL to about 40 μg/μL, or about 0.5 μg/μL to about 20 μg/μL, or about 0.5 μg/μL to about 10 μg/μL.

In one example, sample containing rare nucleic acids or rare cells is collected into a container and mixed with a suitable buffer. The collected sample is subjected to filtration to concentrate the number of rare nucleic acids or rare cells with rare nucleic acids. In another example, nucleic acid binding matrix with a cell affinity agent is used to selectively bind to a rare cell. In another example, a nucleic acid binding matrix with a nucleic acid affinity agent is used to selectively bind to a rare nucleic acid. In another example, the nucleic acid binding matrix with a cell affinity agent and nucleic acid affinity agent are combined with the sample and the rare cells and rare nucleic are retained on a porous matrix of a filtration device. After a suitable incubation period, the matrix is washed with a buffer.

After unbound nucleic acid and cells are washed away from the porous matrix, the nucleic acids retained on the porous matrix are washed away. In case of rare cell, unbound cells are lysed and lysates collected. The nucleic acid sample is collected into a container with a suitable buffer. At this point the rare nucleic acid is non-cellular, i.e., the rare nucleic acid is not bound to a cell. The collected sample is combined with additional nucleic acids reagent to allow amplification and analysis.

### Examples of Porous Matrix

The porous matrix is a solid material, which is impermeable to liquid except through one or more pores of the matrix in accordance with the principles described herein. The porous matrix may be comprised of an organic or inorganic, water insoluble material. The porous matrix is associated with a porous matrix holder and a liquid holding well. The association between porous matrix and holder can be done with an adhesive. The association between porous matrix in the holder and the liquid holding well can be through direct contact or with a flexible gasket surface.

The porous matrix is non-bibulous, which means that the porous matrix is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the porous matrix is at least 95% free of fibers, or at least 99% free of fibers, or at least 99.5%, or at least 99.9% free of fibers, or 100% free of fibers.

The porous matrix can have any of a number of shapes such as, for example, track-etched, or planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the nature or shape of holder for the porous matrix, of the microfluidic surface, of the liquid holding area, of cover surface, for example. In some examples the shape of the porous matrix is circular, oval, rectangular, square, track-etched, planar or flat surface (e.g., strip, disk, film, membrane, and plate).

The porous matrix may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly (vinyl chloride), polyacrylamide, polyacrylate, polyethylene, polypropylene, poly-(4-methylbutene), polystyrene, polymethacrylate, poly(ethylene terephthalate), nylon, poly(vinyl butyrate), poly(chlorotrifluoroethylene), poly(vinyl butyrate), polyimide, polyurethane, and parylene; silanes; silicon; silicon nitride; graphite; ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and PYREX®); and bioresorbable polymers (such as, e.g., polylactic acid, polycaprolactone and polyglycoic acid); either used by themselves or in conjunction with one another and/or with other materials. The material for fabrication of the porous matrix and holder are non-bibulous and does not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The material for fabrication of the porous matrix and holder may be the same or different materials.

The porous matrix for each liquid holding area comprises at least one pore and no more than about 2,000,000 pores per square centimeter (cm²). In some examples the number of pores of the porous matrix per cm² is 1 to about 2,000,000, or 1 to about 1,000,000, or 1 to about 500,000, or 1 to about 200,000, or 1 to about 100,000, or 1 to about 50,000, or 1 to about 25,000, or 1 to about 10,000, or 1 to about 5,000, or 1 to about 1,000, or 1 to about 500, or 1 to about 200, or 1 to about 100, or 1 to about 50, or 1 to about 20, or 1 to about 10, or 2 to about 500,000, or 2 to about 200,000, or 2 to about 100,000, or 2 to about 50,000, or 2 to about 25,000, or 2 to about 10,000, or 2 to about 5,000, or 2 to about 1,000, or 2 to about 500, or 2 to about 200, or 2 to about 100, or 2 to about 50, or 2 to about 20, or 2 to about 10, or 5 to about 200,000, or 5 to about 100,000, or 5 to about 50,000, or 5 to about 25,000, or 5 to about 10,000, or 5 to about 5,000, or 5 to about 1,000, or 5 to about 500, or 5 to about 200, or 5 to about 100, or 5 to about 50, or 5 to about 20, or 5 to about 10, for example. The density of pores in the porous matrix is about 1% to about 20%, or about 1% to about 10%, or about 1% to about 5%, or about 5% to about 20%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein. The size of the pores of the porous matrix is dependent on the nature of the liquid, the size of the cell, the size of the capture particle, the size of mass label, the size of an analyte, the size of label particles, the size of non-rare molecules, and the size of non-rare cells, for example. In some examples the average size of the pores of the porous matrixes are about 0.1 to about 20 microns, or about 0.1 to about 5 microns, or about 0.1 to about 1 micron, or about 1 to about 20 microns, or about 1 to about 5 microns, or about 1 to about 2 microns, or about 5 to about 20 microns, or about 5 to about 10 microns.

Pores within the matrix may be fabricated in accordance with the principles described herein by, for example, microelectromechanical (MEMS) technology, metal oxide semiconductor (CMOS) technology, micro-manufacturing processes for producing micro-sieves, laser technology, irradiation, molding, and micromachining, for example, or a combination thereof.

The porous matrix is attached to a liquid holding area. In some examples the porous matrix is permanently fixed to a liquid holding area by an adhesive or bonding method. The porous matrix permanently fixed to a liquid holding area is associated with a microfluidic surface. In other examples the porous matrix is permanently fixed to a porous matrix “holder” which is associated with the liquid holding area and microfluidic surface. The porous matrix can be associated to the bottom of the liquid holding area and top of microfluidic surface by means of force or fit with or without use of a gasket.

The porous matrix may be permanently attached to a holder by adhesive or bonding method such as ultrasonic bonding, UV bonding, thermal bonding, mechanical fastening or through use of permanently adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein and lignin. The plastic or the adhesive can be electrically conductive materials and the conductive material coatings or materials can be patterned across specific regions of the hold surface.

Examples of plastic film materials include polystyrene, polyalkylenes, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently fixed attached to a holder by adhesion using thermal bonding, mechanical fastening or through use of permanently adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein and lignin. The plastic film or the adhesive can be electrically conductive materials and the conductive material coatings or materials can be patterned across specific regions of the hold surface.

The porous matrix in the holder is generally part of a filtration module where the porous matrix is part of an assembly for convenient use during filtration. The holder does not contain pores and has a surface which facilitates contact with associated surfaces but is not permanently attached to these surfaces and can be removed. A top gasket maybe applied to the removable holder between the liquid holding wells. A bottom gasket maybe applied to the removable holder between the manifold for vacuum. A gasket is a flexible material that facilities complete contact upon compression. The holder maybe constructed of gasket material. Examples of gasket shapes include a flat, embossed, patterned, or molded sheets, rings, circles, ovals, with cut out areas to allow sample to flow from porous matrix to vacuum maniford. Examples of gasket materials include paper, rubber, silicone, metal, cork, felt, neoprene, nitrile rubber, fiberglass, polytetrafluoroethylene like PTFE or Teflon or a plastic polymer like polychlorotri-fluoroethylene.

In some examples, vacuum is applied to the concentrated and treated sample on the porous matrix to facilitate passage of non-rare cells through the matrix. The level of vacuum applied is dependent on one or more of the nature and size of the different populations of biological particles, the nature of the porous matrix, and the size of the pores of the porous matrix. In some examples, the level of vacuum applied is about 1 millibar to about 100 millibar, or about 1 millibar to about 80 millibar, or about 1 millibar to about 50 millibar, or about 1 millibar to about 40 millibar, or about 1 millibar to about 30 millibar, or about 1 millibar to about 25 millibar, or about 1 millibar to about 20 millibar, or about 1 millibar to about 15 millibar, or about 1 millibar to about 10 millibar, or about 5 millibar to about 80 millibar, or about 5 millibar to about 50 millibar, or about 5 millibar to about 30 millibar, or about 5 millibar to about 25 millibar, or about 5 millibar to about 20 millibar, or about 5 millibar to about 15 millibar, or about 5 millibar to about 10 millibar. In some examples, the vacuum is an oscillating vacuum, which means that the vacuum is applied intermittently at regular or irregular intervals, which may be, for example, about 1 second to about 600 seconds, or about 1 second to about 500 seconds, or about 1 second to about 250 seconds, or about 1 second to about 100 seconds, or about 1 second to about 50 seconds, or about 10 seconds to about 600 seconds, or about 10 seconds to about 500 seconds, or about 10 seconds to about 250 seconds, or about 10 seconds to about 100 seconds, or about 10 seconds to about 50 seconds, or about 100 seconds to about 600 seconds, or about 100 seconds to about 500 seconds, or about 100 seconds to about 250 seconds. In this approach, vacuum is oscillated at about 0 millibar to about 10 millibar, or about 1 millibar to about 10 millibar, or about 1 millibar to about 7.5 millibar, or about 1 millibar to about 5.0 millibar, or about 1 millibar to about 2.5 millibar, for example, during some or all of the application of vacuum to the blood sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes.

### Examples of Capture Particle

As mentioned above, the nucleic acid binding matrix maybe a capture particle that includes nucleic acid affinity agents, cell affinity agents, or hybridization oligo or combinations thereof. The capture particle can have nucleic acid affinity agents that are specific for one or more rare nucleic acid, or non-specifically binding to all nucleic acids or selective binding to certain types of nucleic acids. The capture particle can have cell affinity agents that are specific for one or more rare cells, or non-specifically binding to all rare cells or selective binding to certain types of rare cells. The capture particles can be prepared by directly attaching nucleic acid affinity agents, cell affinity agents, or hybridization oligo, individually to different capture particle. The capture particle can be multiplexed for more than one result at a time. Alternatively, different capture particles and different affinity agents or oligos can be combined and reacted. The nucleic acid affinity agent, cell affinity agent or hybridization oligo can be attached to separate capture particle. The nucleic acid affinity agent, cell affinity agent or hybridization oligo can be bound to one capture particle.

The composition of the capture particle may be, for example, as described above for capture particle entities. The size of the capture particle is large enough to accommodate one or more affinity agent or oligo. The ratio of affinity agents or oligo to a single capture particle may be 10⁷ to 1, 10⁶ to 1, or 10⁵ to 1, or 10⁴ to 1, or 10³ to 1, or 10² to 1, or 10 to 1. The number of affinity agent or oligo associated with the label particle is dependent on one or more of the nature and size of the affinity agent or oligo, the nature and size of the label particle, the nature of the linker arm, the number and type of functional groups on the label particle, and the number and type of functional groups on the capture particle.

The composition of the capture particle entity may be organic or inorganic, magnetic or non-magnetic as a nanoparticle or a microparticle. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), melamine resin, nylon, poly(vinyl butyrate), either used by themselves or in conjunction with other materials and including latex, microparticle and nanoparticle forms thereof. The particles may also comprise carbon (e.g., carbon nanotubes), metal (e.g., gold, silver, and iron, including metal oxides thereof), colloids, dendrimers, dendrons, and liposomes, for example. In some examples, the label particle may be a silica nanoparticle. In other examples, capture particles can be magnetic that have free carboxylic acid, amine or tosyl groups.

The diameter of the capture particle is dependent on one or more of the nature of the rare molecule, the nature of the sample, the permeability of the cell, the size of the cell, the size of the nucleic acid, the size of the affinity agent, the size of the oligo, the magnetic forces applied for separation, the nature and the pore size of a filtration matrix, the adhesion of the particle to matrix, the surface of the particle, the surface of the matrix, the liquid ionic strength, liquid surface tension and components in the liquid, and the number, size, shape and molecular structure of associated label particles. When a porous matrix is employed in filtration separation step, the diameter of the capture particles must be large enough to hold a number of affinity agents or oligo to achieve the benefits of rare molecule capture and amplification in accordance with the principles described herein but small enough to pass through the pores of a porous matrix or matrix of a filtration device in accordance with the principles described herein.

In some examples in accordance with the principles described herein, the average diameter of the particles should be at least about 0.02 microns (20 nm) and not more than about 10 microns. In some examples, the particles have an average diameter from about 0.02 microns to about 0.06 microns, or about 0.03 microns to about 0.1 microns, or about 0.06 microns to about 0.2 microns, or about 0.2 microns to about 1 micron, or about 1 micron to about 3 microns, or about 3 micron to about 10 microns, In some examples, the adhesion of the particles to the surface is so strong that the particle diameter can be smaller than the pore size of the matrix. In other examples, the particles are sufficiently larger than the pore size of the matrix such that physically the particles cannot fall through the pores.

The capture particles can be bound through “binding partners” or attached through “linking groups” to nucleic acid affinity agents, to the cell affinity agents, or to hybridization oligo. The capture particles can be additionally bound through “binding partners” to other particles, like magnetic particles, or to a surface, like a membrane. The capture particle can contain one member of the “binding partners”. The other member of the binding partners can be included on the nucleic acid affinity agent, the cell affinity agent, the hybridization oligo, additional particle or surface. The phrase “binding partner” refers to a molecule that is a member of a specific binding pair. A member of a specific binding pair is one of two different molecules having an area on the surface or in a cavity, which specifically binds to and is thereby defined as complementary with a particular spatial and polar organization of the other molecule. In some cases, the affinity agent may be members of an immunological pair such as antigen to antibody or hapten to antibody, biotin to avidin, IgG to protein A, secondary antibody to primary antibody, antibodies to fluorescent labels and other examples of binding pairs.

Obtaining reproducibility in amounts of particle captured after separation and isolation is important for rare molecular analysis. Additionally, knowing the amounts of particle captured that bind a rare cell is important to maximize the amount of specific binding. Knowing the amount of remaining particles after washing is important to minimize the amount of non-selective binding. In order to make these determinations, it is helpful if the particles can contain fluorescent labels. Therefore, capture particles, can be measured by fluorescent techniques by virtue of the presence of a fluorescent molecule. The fluorescent molecule can then be measured by microscopic analysis and compared to expected results for sample containing and lacking analyte. Fluorescent molecule include but are not limited to DYLIGHT™, FITC, rhodamine compounds, phycoerythrin, phycocyanin, allophycocyanin, o-phthaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynylanthracene, squaraine dyes and fluorescamine. A fluorescent microscope or fluorescent spectrometer may then be used to determine the location and amount of the capture particles.

The linking group between the capture particle and the affinity agent, affinity label, mass label, hybridization oligo or fluorescent labels may be an aliphatic or aromatic bond. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

The linking group between the capture particle and the affinity agent may be a chain of from 1 to about 60 or more atoms, or from 1 to about 50 atoms, or from 1 to about 40 atoms, or from 1 to 30 atoms, or from about 1 to about 20 atoms, or from about 1 to about 10 atoms, each independently selected from the group consisting of carbon, oxygen, sulfur, nitrogen, and phosphorous, usually carbon and oxygen. The number of heteroatoms in the linking group may range from about 0 to about 8, from about 1 to about 6, or about 2 to about 4. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

One or more linking groups may comprise a cleavable moiety that is cleavable by a cleavage agent. The nature of the cleavage agent is dependent on the nature of the cleavable moiety. Cleavage of the cleavable moiety may be achieved by chemical or physical methods, involving one or more of oxidation, reduction, solvolysis, e.g., hydrolysis, photolysis, thermolysis, electrolysis, sonication, and chemical substitution. Examples of cleavable moieties and corresponding cleavage agents, by way of illustration and not limitation, include disulfide that may be cleaved using a reducing agent, e.g., a thiol; diols that may be cleaved using an oxidation agent, e.g., periodate; diketones that may be cleaved by permanganate or osmium tetroxide; diazo linkages or oxime linkages that may be cleaved with hydrosulfite; β-sulfones, which may be cleaved under basic conditions; tetralkylammonium, trialkylsulfonium, tetra-alkylphosphonium, where the α-carbon is activated, e.g., with carbonyl or nitro, that may be cleaved with base; ester and thioester linkages that may be cleaved using a hydrolysis agent such as, e.g., hydroxylamine, ammonia or trialkylamine (e.g., trimethylamine or triethylamine) under alkaline conditions; quinones where elimination occurs with reduction; substituted benzyl ethers that can be cleaved photolytically; carbonates that can be cleaved thermally; metal chelates where the ligands can be displaced with a higher affinity ligand; thioethers that may be cleaved with singlet oxygen; hydrazone linkages that are cleavable under acidic conditions; quaternary ammonium salts (cleavable by, e.g., aqueous sodium hydroxide); trifluoroacetic acid-cleavable moieties such as, e.g., benzyl alcohol derivatives, teicoplanin aglycone, acetals and thioacetals; thioethers that may be cleaved using, e.g., HF or cresol; sulfonyls (cleavable by, e.g., trifluoromethane sulfonic acid, trifluoroacetic acid, or thioanisole); nucleophile-cleavable sites such as phthalamide (cleavable, e.g., with substituted hydrazines); ionic association (attraction of oppositely charged moieties) where cleavage may be realized by changing the ionic strength of the medium, adding a disruptive ionic substance, lowering or raising the pH, adding a surfactant, sonication, and adding charged chemicals; and photocleavable bonds that are cleavable with light having an appropriate wavelength such as, e.g., UV light at 300 nm or greater.

In one example, a cleavable linkage may be formed using conjugation with N-succinimidyl 3-(2-pyridyldithio)propionate) (SPDP), which comprises a disulfide bond. For example, a label particle comprising an amine functionality is conjugated to SPDP and the resulting conjugate can then be reacted with a nucleic acid affinity agent comprising a thiol functionality, which results in the linkage of the nucleic acid affinity agent moiety to the conjugate. A disulfide reducing agent (such as, for example, dithiothreitol (DTT) or tris(2-carboxyethyl)phosphine (TCEP)) may be employed as a release agent.

### Examples of Nucleic Acids Affinity Agent

A nucleic acid affinity agent is a molecule capable of selectively binding to nucleic acids. Specific binding involves the specific recognition of one of two different molecules for the other compared to substantially less recognition of other molecules. The nucleic acid affinity agent is capable of being absorbed into or onto the cell and associated with a capture particle through a “binding pair” or a direct linkage. The nucleic acid affinity agent can bind selectively to one or more corresponding rare nucleic acids with a common sequence in a population of nucleic acids with different sequence. The nucleic acid affinity agent allows differentiation of one of the populations of rare nucleic acids from other populations of rare nucleic acids and separation to permit multiplexing.

Nucleic acid affinity agents include nucleic acid binding proteins. These proteins include RNA binding proteins and DNA binding nucleic acid binding protein. These proteins also include unreactive helicases, polymerase and nucleases which can bind nucleic acids and not alter the nucleic acids. These proteins can be antibodies that specifically bind nucleic acid such as with single-stranded DNA (ssDNA), and/or double-stranded DNA (dsDNA), Z-DNA, tRNA, rRNA and nucleoproteins like small nuclear ribonucleoproteins (snRNP). Some of these antibodies react with RNA-DNA duplexes where they bind to both RNA and ssDNA. Complementary RNA or ss DNA can be added to cause binding duplex formation and allow antibody binding. Therefore a specific RNA or ssDNA target can be bound by the antibody serving as a nucleic acid affinity agents RNA binding proteins include proteins with RNA binding domains (RBD, also known as RNP domain), or RNA recognition motif (RRM). These include Arg-Gly-Gly (RGG), K-homology domain (KH domain), piwil/argonaute/zwille (PAZ domain), PUsed, Zinc fingers (ZnF), Sm domain, DEAD/DEAH box, cold-shock domain, Pumilio/FBF domain (PUF or Pum-HD), double stranded RNA-binding domain (dsRBD) as well as others. There are at least 1171 RNA-binding proteins in current databases of RNA binding protein databases (http://rbpdb.ccbr.utoronto.ca).

DNA binding proteins include proteins with DNA-binding domains and have a specific or general affinity for either single or double stranded DNA. Sequence-specific DNA-binding proteins generally interact with the major groove of B-DNA, because it exposes more functional groups that identify a base pair. Some DNA-binding proteins specifically bind single-stranded DNA, such as protein A. Other DNA-binding proteins bind to specific DNA sequences, various transcription factors, which are proteins that regulate transcription. DNA binding proteins include helix turn helix, zinc finger, DNA recombinases, leucine zipper, winged helix, turn helix, winged helix turn helix, helix loop helix, HMG-box, HMG box, Wor3 domains, OB fold domains, Immunoglobulin fold, B3 domain, TAL effector DNA binding domains, RNA effortor, DNA binding domains as well as others. There are at least 1013 human DNA-binding proteins in current databases of DNA binding protein databases (http://bioinfo.wilmer.jhu.edu/PDI) as well 493 human transcription factors (TFs) and 520 unconventional DNA binding proteins (uDBPs) which are also human DNA-binding proteins.

### Examples of Hybridization Oligo

The hybridization oligo is a nucleic acid (e.g., polynucleotide) that is complementary to a rare nucleic acid to be detected. It can then be used in DNA or RNA samples to detect the presence of nucleotide sequences (the target) that are complementary to the sequence in the probe. Polynucleotides refer to a polymeric form of nucleotides of any length, either deoxy-ribonucleotides or ribonucleotides, or analogs thereof. A structural feature of the nucleic acid can be exploited for affinity agent. For example virtually all eukaryotic mRNA have a 7-methylguaninie nucleotide linked to its 5′ end and is polyadenylated at the opposite 3′ end by action of poly(A) polymerase. The poly(A) tail is used to purify mRNA by affinity chromatography on oligo(dT) matrix.

In some examples, hybridization techniques may be employed to bind the hybridization oligo to rare nucleic acids that are present on or within a rare cell. In other cases hybridization techniques may be employed to bind the hybridization oligo to rare nucleic acids that are not associated with cells.

As with any other nucleic acid hybridization, the main factors influencing the selectivity of the hybridization oligo are: the amount of repetitive sequences of the oligo hybridization oligo and the extent to which they are blocked from binding from other nucleic acids; the hybridization temperature (lowering it increases nonspecific binding of the repetitive sequences); the balance between hybridization time and amount of hybridization oligo; the stringency of the post-hybridization washes. There are variables to be considered during the post hybridization washessuch as the composition of solutions, washing temperature and the washing time.

The following are non-limiting examples of hybridization oligo, a polynucleotide complementary to the sequence to coding or non-coding regions of a rare nucleic acid. The polynucleotide may comprise modified nucleotides such as, for example, methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The sequence of hybridization oligo may be interrupted by non-nucleotide components. A polynucleotide may be further modified, such as by conjugation with a labeling component. The terms “isolated nucleic acid” and “isolated polynucleotide” are used inter-changeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a larger sequence. Hybridization oligos can be of variable length (usually 15-1000 bases long)

When probing for mRNAs, an RNase treatment step is often added to determine that the binding is specific to RNA by digesting the cells with RNases prior to hybridization with the oligonucleotide probe. The absence of binding after RNase treatment indicates that binding was indeed to RNA within the sample. Another commonly observed pre-treatment when using RNA probes is acetylation with acetic anhydride (0.25%) in triethanolamine. This treatment is thought to be important for decreasing background but it also appears to inactivate RNases and may help in producing a strong signal.

Hybridization and washing chemicals are typically required for any hybridization method. The hybridization process is critical in controlling the efficiency of the probe to anneal to a complementary hybridization oligo whether RNA or DNA strand just below its melting point (Tₘ). The RNA or DNA and the probe can be simultaneously denaturized using a chemical hybridization solution. The probe can be annealed at the melting point along with blocking competitor DNA which might be used as option to reduce non-binding to repetitive sequences. The most common suppressor DNAs tested were Cot1 DNA and salmon sperm DNA. Repetitive sequences (especially Alu and L1 families in human) have to be blocked with competitor DNA prior to FISH. Additional control probe or multiple probes can also be added. Hybridization solution temperatures can be varied from at 25 to 100° C. over time periods of 5 min to 25 hours.

### Examples of Cell Affinity Agent

A cell affinity agent is a molecule capable of binding selectively to rare cells containing nucleic acids. A cell affinity agent is a cell typing marker and selective binding involves the specific recognition of one of two different molecules for the other compared to substantially less recognition of other molecules. Selective cell binding typically involves non-covalent binding between molecules that is relatively dependent of specific structures of binding pair. Selective binding does not rely on non-specific recognition. Non-specific binding may result from several factors including hydrophobic or electrostatic interactions between molecules that are general and not specific to any particular molecule in a class of similar molecules.

A cell affinity agent can be a protein, peptide, glycoconjugate, immunoglobulins, or other marker capable of binding selectively to a particular rare cell type. These rare cell typing markers can be immunoglobulins that specifically recognize and bind to an antigen associated with a particular cell type and whereby antigen are components of the cell. The cell affinity agent is capable of being absorbed into or onto the cell and associated with a capture particle through a “binding pair” or a direct linkage.

Antibodies are specific for a rare cell typing markers and can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, and Fab′, for example. In addition, aggregates, polymers, and conjugates of immunoglobulins or their fragments can be used where appropriate so long as binding affinity for a particular molecule is maintained.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, HPLC chromatography; and filtration.

Examples of Selective Nucleic Acid Amplification Selective amplification refers to replication of rare nucleic acid sequences or segments of the sequences to preferentially increase the total copy numbers of these sequences or sequence segments over non-rare nucleic acid sequences. Such techniques include, but are not limited to, enzymatic amplification such as, for example, polymerase chain reaction (PCR), ligase chain reaction (LCR), nucleic acid sequence based amplification (NASBA), Q-β-replicase amplification, 3 SR (specific for RNA and similar to NASBA except that the RNAase-H activity is present in the reverse transcriptase), transcription mediated amplification (TMA) (similar to NASBA in utilizing two enzymes in a self-sustained sequence replication), whole genome amplification (WGA) with or without a secondary amplification such as, e.g., PCR, multiple displacement amplification (MDA) with or without a secondary amplification such as, e.g., PCR, whole transcriptome amplification (WTA) with or without a secondary amplification such as, e.g., PCR or reverse transcriptase PCR.

The methods must achieve a high fidelity amplification with lowest number of non-rare nucleic acid molecules amplified and contaminating the desired amplified rare nucleic acids as the result of a low error rate in duplicating the rare nucleic acid molecules. The methods described herein involve trace analysis, i.e., minute amounts of material on the order of 100 to about 10,000,000 minimal copy number. Since this process involves trace analysis at the detection limits of the nucleic acid analyzers, these minute amounts of material can only be detected when amplification is on order of about 10⁵ to about 10¹⁰ fold of every rare molecule, so that the concentrations are within the detection limits of nucleic acid analysis.

Given errors associated with multiple amplification, a high fidelity amplification is needed which likely and that “all” of the rare molecules undergo amplification, i.e., converting the minute amounts of material. The phrase “substantially all” means that at least about 98% reproducibly replicated on each amplification cycle producing a new amount of the rare nucleic acid.

Selective amplification must use minimal cycle number to maintain a high fidelity amplification. The minimal cycle number is the lowest number of allowed amplification cycles that are needed for nucleic acids analysis while a high fidelity amplification is maintained. The minimal cycle number is generally on the order of less than 40 amplification cycles for a minimal copy number of rare nucleic acids on the order of 100 to about 10,000,000 minimal copy number. In some examples, the minimal cycle number is 10 to about 20 cycles, or is 10 to about 30 cycles, or is 30 to about 40 cycles. After amplification, the sample can be split into more than one aliquot and the aliquots can be removed for nucleic acid corrected analysis.

High fidelity amplification of nucleic acid sequences or select regions of the sequences may be carried out by any suitable methods. Examples of suitable amplification methods include, but not limited to, polymerase chain reaction (PCR) (U.S. Pat. Nos. 4,683,195; 4,683,202; and 4,965,188), ligase chain reaction (LCR) (Wiedmann M, et al., PCR Methods Appl. 1994; 3(4):551-64), loop mediated isothermal amplification (LAMP) (Notomi T, et al., Nucleic Acids Research. 2000; 28(12):e63.), multiple displacement amplification (MDA) (Paez J G, et al., Nucleic Acids Research. 2004; 32(9):e71), nucleic acid sequence-based amplification (NASBA) (Compton J, Nature. 1991; 350(6313):91-2), helicase-dependent amplification (HDA) (Saiki R K, et al., Science 1988; 239(4839):487-91), rolling circle amplification (RCA) (Ali M M, et al., Chem Soc Rev. 2014; 43(10):3324-41), recombinase polymerase amplification (RPA) (Piepenburg O, et al., PLoS Biol. 2006; 4(7):e204). Polymerase chain reaction is generally the preferred method for nucleic acid amplification. Ribonucleic acid (RNA) sequences are usually first converted to complementary DNA (cDNA) sequences through reverse transcription followed by amplification of the cDNA using suitable amplification methods.

### Examples of Nucleic Acid Analysis

In all examples after amplification, the sample can be used to perform a nucleic acid corrected analysis. Following extraction of the nucleic acids from the rare cells or nucleic acid binding matrix and selective amplification, the rare nucleic acids are subjected to one or more nucleic acid analysis techniques for quantitation, identification or determination of the rare nucleic acids. Nucleic acid analysis can also be carried out by determining the sequences of the nucleic acids in the sample and comparing them to expected sequences. Nucleic acid sequencing can be done with any suitable sequencing methods. Suitable sequencing methods include but not limited to traditional sequencing methods such as chain-termination based Sanger sequencing (Sanger F, et al., Proc Natl Acad Sci USA. 1977; 74(12):5463-7), and high-throughput sequencing methods (Goodwin S, et al., Nat Rev Genet. 2016 May 17; 17(6):333-51) such as sequencing by synthesis (Illumina), sequencing by ligation (SOLID), ion semiconductor sequencing (Ion Torrent), single-molecule real-time sequencing (Pacific Biosciences), and nanopore sequencing. Matching or aligning acquired sequences to expected target sequences can be used to confirm the presence of target nucleic acids, while the number of correct sequence copies can be used to quantify target nucleic acids.

Analysis of nucleic acids can be achieved by using molecular tags or labels that can generate physical or chemical signals, including but not limited to fluorescent, luminescent, electrical, and radioactive signals. A signaling tag such as a fluorescent dye or a radioisotope label can be covalently linked to the target nucleic acid, or non-covalently intercalate into the target nucleic acid strand to produce measurable signals. In other cases, a nucleic acid probe that is complementary to the target nucleic acid is labeled with a signaling tag. After binding to the target nucleic acid by the probe and removing unbound probe, the measured signals from the probe can be used to detect and quantify the target nucleic acid. A nucleic acid separation method, including but not limited to gel electrophoresis, capillary electrophoresis, and microfluidic channels, can be used to separate target nucleic acid from other nucleic acids based on mobility prior to signal measurements to verify correct size of the target nucleic acid.

In other cases, signal generation for nucleic acid analysis can happen during nucleic acid amplification such as in real-time PCR. An example of this is the use of DNA intercalating dyes. These fluorescent dyes can be incorporated into the double-stranded DNA amplification products which induces enhanced fluorescent signals. In another example, a nucleic acid probe is labeled with a fluorescent dye and quencher on the same strand, and is complementary to a segment of the nucleic acid sequence that is being amplified. During amplification, the probe can bind to its complementary strand. As the polymerase synthesizes and extends on the same strand, it can cleave the probe bound to the strand and release the fluorescent dye from the quencher, which produces enhanced fluorescent signals. Signals generated during amplification can be used to quantify target nucleic acids.

In corrected nucleic acids analysis, rare nucleic acids analyzed are a combination of disease-related nucleic acids and reference nucleic acids. The disease-related nucleic acids are nucleic acids that allows for distinguishing an abnormal condition from the normal condition. The reference nucleic acids are nucleic acids that are present in both rare cells and non-rare cells at similar level. Disease-related nucleic acids are corrected by ratio of disease-related nucleic acids to reference nucleic acids for determining whether the rare disease-related nucleic acid is present. If the corrected detection of rare disease-related nucleic acid rare nucleic acid are present, then the sample could be flagged for additional analysis such as sequencing, expression analysis, or quantitation.

Subsequent to identification, the nucleic acids can be subjected to further analytic techniques such as, but not limited to, sequencing techniques, PCR, branched DNA testing, ligase chain reaction, and hybridization methods, including combinations of two or more of the above. Methods of sequencing nucleic acids include, by way of illustration and not limitation, chemical sequencing (e.g., Maxam-Gilbert sequencing), chain termination sequencing (e.g., Sanger sequencing), de novo sequencing, shotgun sequencing, in vitro clonal amplification (e.g., bridge PCR), high throughput sequencing, sequencing by ligation (SOLID sequencing), sequencing by synthesis, pyrosequencing, ion semiconductor sequencing, single molecule real-time sequencing, massively parallel signature sequencing (MPSS), Polony sequencing, DNA nanoball sequencing, single molecule sequencing, and combinations thereof.

Identification agents for identifying nucleic acids include, by way of illustration and not limitation, nucleic acid probes that have sequences complementary to sequences of nucleic acids (and are, therefore, specific for the complementary sequence). The nucleic acid probe may be, or may be capable of being, labeled with a reporter group (a label), or may be capable of becoming, bound to a support, or both. Binding of the probes to nucleic acid sequences is detected by means of the labels. Binding can be detected by separating the bound probe from the free probe and detecting the label. In one example, a sandwich is formed comprised of the labeled probe, the sequence and a probe that is or can become bound to a surface. Alternatively, binding can be detected by a change in the signal-producing properties of the label upon binding off the probe with the sequence, such as a change in the emission efficiency of a fluorescent or chemiluminescent label. This permits detection to be carried out without a separation step. Detection of signal depends upon the nature of the label or reporter group. If the label or reporter group is an enzyme, additional members of the signal producing system include, for example, enzyme substrates. In one approach the nucleic acids are immobilized on a solid support and then contacted with suitable labeled nucleic acid probes followed by detection of the labels.

The label is usually part of a signal producing system, which includes one or more components, at least one component being a detectable label, which generates a detectable signal that relates to the amount of bound and/or unbound label, i.e. the amount of label bound or not bound to the nucleic acid being detected or to an agent that reflects the amount of the nucleic acid to be detected. The label is any molecule that produces or can be induced to produce a signal, and may be, for example, a fluorophore, a radiolabel, an enzyme, a chemiluminescent agent or a photosensitizer. Thus, the signal is detected and/or measured by detecting enzyme activity, luminescence, light absorbance or radioactivity, depending on the nature of the label. Suitable labels include, by way of illustration and not limitation, dyes; fluorophores, such as fluorescein, isothiocyanate, rhodamine compounds, phycoerythrin, phycocyanin, allophycocyanin, o-phthalaldehyde, and fluorescamine; enzymes such as alkaline phosphatase, glucose-6-phosphate dehydrogenase (“G6PDH”), β-galatosidase, and horseradish peroxidase; ribozyme; a substrate for a replicase such as QB replicase; promoters; complexes such as those prepared from CdSe and ZnS present in semiconductor nanocrystals known as Quantum dots; chemiluminescent agents such as isoluminol and acridinium esters, for example; sensitizers; coenzymes; enzyme substrates; radiolabels such as ³²P, ¹²⁵I, ¹³¹I, ¹⁴C, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group.

The label can directly produce a signal and, therefore, additional components are not required to produce a signal. Numerous organic molecules, for example fluorophores, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

### Examples of Rare Nucleic Acids

The following are non-limiting examples of rare nucleic acids such as coding or non-coding regions of a gene or gene fragment, loci (locus) defined from linkage analysis, exons, introns, messenger RNA (mRNA), transfer RNA, ribosomal RNA, ribozymes, cDNA, circulating DNA/RNA, recombinant polynucleotides, branched polynucleotides, plasmids, vectors, isolated DNA of any sequence, isolated RNA of any sequence, nucleic acid probes, and primers.

The sample to be analyzed is one that is suspected of containing rare nucleic acid. The samples may be biological samples or non-biological samples. Samples include solutions, mixtures and slurries. The samples may be from cells, plants, soils, solution, cultures, from the production of biochemical, cell and chemical production and from feed stocks for plants, organisms, production process or mammalian subjects or waste streams for plants, organisms, production process or mammalian subjects. Samples can be from mammalian subjects or a non-mammalian subjects. In many instances, the sample is used in agriculture, biotechnology processes, geological process, mining process. from ground water, drinking water and the like. Biological samples from mammalian subjects may be, e.g., humans or any other animal species. Biological samples include biological fluids such as whole blood, serum, plasma, sputum, lymphatic fluid, semen, vaginal mucus, feces, urine, spinal fluid, saliva, stool, cerebral spinal fluid, tears, and mucus.

In some examples of methods in accordance with the principles described herein, the sample suspected of containing rare nucleic acid to be tested are a biological sample. In some examples of methods in accordance with the principles described herein, the sample to be tested is a biological sample from a mammal, cell, plant, organism and the like. Biological samples may contain rare nucleic acids from tissue and parts of tissue including, by way of illustration, hair, skin, sections or excised tissues from organs or other body parts. Rare nucleic acid may be from, for example, lung, bronchus, colon, rectum, pancreas, prostate, breast, liver, bile duct, bladder, ovary, brain, central nervous system, kidney, pelvis, uterine corpus, oral cavity or pharynx or melanoma cancers.

The rare nucleic acid may be bound in a cell as cellular rare nucleic acids or maybe freely circulate in a sample as cell free rare nucleic acids. Rare nucleic acid cells may be separated from tissues such as malignant neoplasms or cancer cells; circulating endothelial cells; circulating tumor cells; circulating cancer stem cells; circulating cancer mesochymal cells; circulating epithelial cells; progenitor cells, stem cells, fetal cells or from other cells in the biological sample such as pathogens like bacteria, virus, fungus, and protozoa, immune cells (B cells, T cells, macrophages, NK cells, monocytes) and stem cells. The biological sample can contain a mixture of cells such as, for example, non-rare cells and rare cells. The rare nucleic acid may be from non-rare cells and rare cells. The rare nucleic acid may be bound in a biological compartment such as extracellular vesicles, exosomes, viruses, micro-vesicles, apoptotic body, endosomes, lysosomes, cytosomes, cells, and artificial compartments like beads, and droplets.

These rare nucleic acids can be disease-related nucleic acids and can be reference nucleic acids. Disease-related nucleic acids are nucleic acids which changes in expression, nature or sequence during an abnormal condition and can be distinguished from the normal condition. Reference nucleic acids are nucleic acids which do not change in expression, nature or sequence during an abnormal condition and can be distinguished from the abnormal condition. The disease-related nucleic acids are useful in medical diagnosis of diseases, identification of agricultural issues, identification of potential biological threat to organisms, identification of potential production issues and other applications For example, rare nucleic acids include biomolecules useful in medical diagnosis of diseases, which include, but are not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, diabetes, liver diseases, infectious diseases and other biomolecules useful in medical diagnosis of diseases, for example.

Rare nucleic acids of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemokine (C—C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin, LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage inflammatory protein, MMP Matrix metalloproteinase, MPO Myeloperoxidase, mTOR Mammalian of rapamycin, NADH Nicotinamide adenine dinucleotide, NGF Nerve growth factor, NFκB Nuclear factor kappa-light-chain-enhancer of activated B cells, NGAL Neutrophil gelatinase lipocalin, NOS Nitric oxide synthase NOX NADPH oxidase NPY Neuropeptide Yglucose, insulin, proinsulin, c peptide OHdG Hydroxydeoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidylserine, PR Proteinase, PYY Neuropeptide like peptide Y, RAGE Receptors for AGE, ROS Reactive oxygen species, 5100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factor-β, TIMP Tissue inhibitor of metalloproteinases, TNF α Tumor necrosis factors-α, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare nucleic acids of interest that are highly expressed by pancreas include but are not limited to INS insulin, GLU gluogen, NKX6-1 transcription factor, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymotrypsinogen B1 Intracellular, CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), and CTFR cystic fibrosis transmembrane conductance regulator. Rare nucleic acids of interest that are highly expressed by adipose tissue include but are not limited to ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute carrier family 29 (equilibrative nucleoside transporter), member 4 SLC7A10 Solute carrier family 7 (neutral amino acid transporter light chain, asc system), member 10, SPX Spexin hormone and TIMP4 TIMP metallopeptidase inhibitor 4.

Rare nucleic acids of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adreno-corticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare nucleic acids of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Rare nucleic acids of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1 glutamate receptor ionotropic, N-methyl D-aspartate 1, OMG oligodendrocyte myelin glycoprotein, SLC17A7 solute carrier family 17 (vesicular glutamate transporter), member 7, Clorf6l chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare nucleic acids of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyrotropin-releasing hormone.

Rare nucleic acids of interest that are highly expressed by the gastrointestinal tract, salivary gland, esophagus, stomach, duodenum, small intestine, or colon include but are not limited to GKN1 Gastrokine 1, GIF Gastric intrinsic factor (vitamin B synthesis), PGA5 Pepsinogen 5 group I (pepsinogen A), PGA3 Pepsinogen 3, group I (pepsinogen A, PGA4 Pepsinogen 4 group I (pepsinogen A), LCT Lactase, DEFA5 Defensin, alpha 5 Paneth cell-specific, CCL25 Chemokine (C—C motif) ligand 25, DEFA6 Defensin alpha 6 Paneth cell-specific, GAST Gastrin, MS4A10 Membrane-spanning 4-domains subfamily A member 10, ATP4A and ATPase, H+/K+ exchanging alpha polypeptide.

Rare nucleic acids of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Rare nucleic acids of interest that are highly expressed by kidney include but are not limited to UMOD uromodulin, TMEM174 transmembrane protein 174, SLC22A8 solute carrier family 22 (organic anion transporter) member 8, SLC12A1 solute carrier family 12 (sodium/potassium/chloride transporter) member 1, SLC34A1 solute carrier family 34 (type II sodium/phosphate transporter) member 1, SLC22A12 solute carrier family 22 (organic anion/urate transporter) member 12, SLC22A2 solute carrier family 22 (organic cation transporter) member 2, MCCD1 mitochondrial coiled-coil domain 1, AQP2 aquaporin 2 (collecting duct), SLC7A13 solute carrier family 7 (anionic amino acid transporter) member 13, KCNJ1 potassium inwardly-rectifying channel, subfamily J member 1 and SLC22A6 solute carrier family 22 (organic anion transporter) member 6.

Rare nucleic acids of interest that are highly expressed by lung include but are not limited to SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare nucleic acids of interest that are highly expressed by the liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6.

Rare nucleic acids of interest that are highly expressed by the testis or prostate include but are not limited to PRM2 protamine 2, PRM1 protamine 1, TNP1 transition protein 1 (during histone to protamine replacement) TUBA3C tubulin, alpha 3c LELP1 late cornified envelope-like proline-rich 1, BOD1L2 biorientation of chromosomes in cell division 1-like 2, ANKRD7 ankyrin repeat domain 7, PGK2 phosphoglycerate kinase 2, AKAP4 A kinase (PRKA) anchor protein 4, TPD52L3 tumor protein D52-like 3, UBQLN3 ubiquilin 3 and ACTL7A actin-like 7A.

### Examples of Rare Cells Containing Nucleic Acids

Rare cells are those cells that are present in a sample in relatively small quantities when compared to the amount of non-rare cells in a sample and contain nucleic acids. In some examples, the rare cells are present in an amount of about 10⁻⁸% to about 10⁻²% by weight of a total cell population in a sample suspected of containing the rare cells. The rare cells may be, but are not limited to, malignant cells such as malignant neoplasms or cancer cells; circulating cells, endothelial cells (CD146); epithelial cells (CD326/EpCAM); mesochymal cells (VIM), bacterial cells, virus, skin cells, sex cells, fetal cells; immune cells (leukocytes such as basophil, granulocytes (CD66b) and eosinophil, lymphocytes such as B cells (CD19,CD20), T cells (CD3,CD4 CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or other for example.

The phrase “population of cells” refers to a group of cells having an antigen or nucleic acid on their surface or inside the cell where the antigen is common to all of the cells of the group and where the antigen is specific for the group of cells. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells.

The term “rare cells markers” include, but are not limited to, cancer cell type biomarkers, cancer biomarkers, chemo resistance biomarkers, metastatic potential biomarkers, cell typing markers, and cluster of differentiation (cluster of designation or classification determinant) (often abbreviated as CD, is a protocol used for the identification and investigation of cell surface molecules providing targets for immunophenotyping of cells). Cancer cell type biomarkers include, by way of illustration and not limitation, cytokeratins (CK) (CK1, CK2, CK3, CK4, CK5, CK6, CK7, CK8 and CK9, CK10, CK12, CK 13, CK14, CK16, CK17, CK18, CK19 and CK20), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CA1X, MIB1, MDM, PR, ER, SELS, SEMI, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDH1, NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ, MET, RB1, AKT1, BRAF, DDR2, MEK1, NRAS, FGFR1, and ROS1.

In certain embodiments, the rare cells may be endothelial cells which are detected using markers, by way of illustration and not limitation such as CD136, CD105/Endoglin, CD144/VE-cadherin, CD145, CD34, Cd41 CD136, CD34, CD90, CD31/PECAM-1, ESAM, VEGFR2/Fik-1, Tie-2, CD202b/TEK, CD56/NCAM, CD73/VAP-2, claudin 5, Z0-1, and vimentin. Metastatic potential biomarkers include, but are not limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARα), plasminogen activator inhibitor (PAI-1), CD95, serine proteases (e.g., plasmin and ADAM, for example), serine protease inhibitors (e.g., Bikunin), matrix metalloproteinases (e.g., MMP9), matrix metalloproteinase inhibitors (e.g., TIMP-1) and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7, CCR7, CXCR4), ESA, CD20, CD44, CD133, CKS, TRAF2 and ABC transporters, cancer cells that lack CD45 or CD31 but contain CD34 are indicative of a cancer stem cell; and cancer cells that contain CD44 but lack CD24.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11c, CD14, CD15, CD16, CD19, CD20, CD31, CD33, CD45, CD52, CD56, CD 61, CD66b, CD123, CTLA-4, immunoglobulin, protein receptors and cytokine receptors and other CD marker that are present on white blood cells can be used to indicate that a cell is not a rare cell of interest. In a particular non-limiting example, CD45 antigen (also known as protein tyrosine phosphatase receptor type C or PTPRC) and originally called leukocyte common antigen is useful in detecting all white blood cells.

Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+, CD3+, CD4+, cytotoxic T cells are indicated by CD45+, CD3+, CDS+, B-lymphocytes are indicated by CD45+, CD19+ or CD45+, CD20+, thrombocytes are indicated by CD45+, CD61+ and natural killer cells are indicated by CD16+, CD56+, and CD3-. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all marker and are for example only.

In other cases the rare cell maybe a stem cell and include but are not limited to markers for stem cells including, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sall4, Stat3, SUZ12, TCF3, TRA-1-60, CDX2, DDX4, Miwi, Mill GCNF, Oct4, Klf4, Sox2,c-Myc, TIF 1βPiwil, nestin, integrin, notch, AML, GATA, Esrrb, Nr5a2, C/EBPα, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii. Faecalibacterium prauznitzli, Lactobacillus gasseria, Streptococcus mutans, Bacteroides thetaiotaomicron, Prevotella Intermedia, Porphyromonas gingivalis, Eubacterium rectale, Lactobacillus amylovorus, Bacillus subtilis, Bifidobacterium longum, Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties, (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MERSA, for example; and sexually transmitted diseases. In the case of detecting rare cell pathogens, a particle reagent is added that comprises a binding partner, which binds to the rare cell pathogen population. Additionally, for each population of cellular rare molecules on the pathogen, a reagent is added that comprises a binding partner for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

Biological samples include biological fluids such as whole blood, serum, plasma, sputum, lymphatic fluid, semen, vaginal mucus, feces, urine, spinal fluid, saliva, stool, cerebral spinal fluid, tears, and mucus, for example. Biological tissue includes, by way of illustration, hair, skin, sections or excised tissues from organs or other body parts, for example. In many instances, the sample is whole blood, plasma or serum. Rare cells may be from, for example, lung, bronchus, colon, rectum, pancreas, prostate, breast, liver, bile duct, bladder, ovary, brain, central nervous system, kidney, pelvis, uterine corpus, oral cavity or pharynx or melanoma cancers. In some examples of methods in accordance with the principles described herein, the sample to be tested is a blood sample from a mammal such as, but not limited to, a human subject, for example. The blood sample is one that contains cells such as, for example, non-rare cells and rare cells. In some examples the blood sample is whole blood or plasma.

### Examples of Reagents for Nucleic Acid Analysis

Depending on method for analysis of rare nucleic acids selected, reagents discussed in more detail herein below, may or may not be used to treat the samples during, prior or after the extraction of nucleic acids from the rare cells and cell free samples. In the event extraction is carried out, a method employed for extraction of nucleic acids from the rare cells is dependent on the nature of the nucleic acids (e.g., DNA or RNA). Extraction of nucleic acids from the rare cells may involve one or more of the following processes: cell lysis; denaturation of DNA and proteins using denaturation agents such as, by way of illustration and not limitation, DNase and proteinase K, for example; removal of cellular membrane lipids; removal of cellular proteins; isolation of nucleic acids onto silica; sucrose gradient modification; spin column centrifugation; chromatography; magnetic particle separations such as, by way of example and not limitation, iron oxide beads coated with a layer of silica, for example; guanidinium acid-phenol extraction; treatment with chaotropic agents such as, but not limited to, guanidinium chloride and guanidinium isothiocyanate, for example; density gradient centrifugation using cesium chloride or cesium trifluoroacetate; use of glass fiber filters; lithium chloride and urea isolation; oligo(dt)-cellulose column chromatography; and non-column poly (A)+ purification/isolation nucleic acid purification.

Cell lysis reagents are used for disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations of two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of the cells to release contents of the cells. In some examples, the amount of the lytic agent is (percentages are by weight) about 0.0001% to about 0.5%, about 0.001% to about 0.4%, about 0.01% to about 0.3%, about 0.01% to about 0.2%, about 0.1% to about 0.3%, about 0.2% to about 0.5% and about 0.1% to about 0.2%.

Removal of lipids may be carried out using, by way of illustration and not limitation, detergents, surfactants, solvents, and binding agents, and combinations of two or more of the above, for example, and combinations of two or more thereof. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples, the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, it may be desirable to remove or denature proteins from the cells, which may be accomplished using a proteolytic agent such as, but not limited to, proteases, heat, acids, phenols, and guanidinium salts, and combinations of two or more thereof. The amount of the proteolytic agent is at least sufficient to degrade at least about 50%, or at least about 60%, or at least about 70%, or at least about 80%, or at least about 90%, or at least about 95% of proteins in the cells. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5% and about 0.1% to about 0.2%.

Methods employed for purifying nucleic acids from the rare cells are chosen based on the nature of the nucleic acids (DNA or RNA). Purification of nucleic acids from the sample as treated above may be carried out using, by way of illustration and not limitation, alcohol precipitation (e.g., ethanol or isopropanol, or a combination thereof) or chloroform precipitation at a temperature of about −10° C. to about 10° C., phenol-chloroform extraction, mini-column purification, affinity chromatography, and magnetic capture, and combinations of two or more thereof. In some examples, samples are collected from the body of a subject into a suitable container such as, but not limited to, a cup, a bag, a bottle, capillary, or a needle, for example.

Blood samples may be collected into VACUTAINER® containers, for example. The container may contain a collection medium into which the sample is delivered. The collection medium is usually a dry medium and may comprise an amount of platelet deactivation agent effective to achieve deactivation of platelets in the blood sample when mixed with the blood sample. Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes.

In many examples, the above combination is provided in an aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples the pH of the aqueous medium is about 5 to about 8, or about 6 to about 8, or about 7 to about 8, or about 5 to about 7, or about 6 to about 7, or physiological pH. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 5 mL to about 100 mL, or about 5 mL to about 80 mL, or about 5 mL to about 60 mL, or about 5 mL to about 50 mL, or about 5 mL to about 30 mL, or about 5 mL to about 20 mL, or about 5 mL to about 10 mL, or about 10 mL to about 100 mL, or about 10 mL to about 80 mL, or about 10 mL to about 60 mL, or about 10 mL to about 50 mL, or about 10 mL to about 30 mL, or about 10 mL to about 20 mL, or about 20 mL to about 100 mL, or about 20 mL to about 80 mL, or about 20 mL to about 60 mL, or about 20 mL to about 50 mL, or about 20 mL to about 30 mL.

Where one or more of the rare nucleic acids are part of a cell, the aqueous medium may also comprise a lysing agent for lysing of cells. A lysing agent is a compound or mixture of compounds that disrupt the integrity of the matrixes of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis. Various ancillary materials may be present in the dilution medium. All of the materials in the aqueous medium are present in a concentration or amount sufficient to achieve the desired effect or function.

In some examples, it may be desirable to fix the nucleic acids or cells of the sample. Fixation immobilizes the nucleic acids and preserves the nucleic acids structure and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both.

In some examples utilizing the techniques, it may be necessary to subject the rare cells to permeabilization. The term “permeability” means the ability of a particles and molecule to enter or exit a cell through the cell wall. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells. In some examples, the amount of permeabilization agent by weight is about 0.1% to about 0.5%, or about 0.1% to about 0.4%, or about 0.1% to about 0.3%, or about 0.1% to about 0.2%, or about 0.2% to about 0.5%, or about 0.2% to about 0.4%, or about 0.2% to about 0.3%. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20). One or more washing steps may be carried out on the permeabilized cells using a buffered aqueous medium.

### Kits for Conducting Methods

The apparatus and reagents for conducting a method in accordance with the principles described herein may be present in a kit useful for conveniently performing the method. In one embodiment a kit comprises in packaged combination of modified capture particles, a nucleic acid affinity agent for each different rare nucleic acid to be isolated. The kit may also comprise one or more cell affinity agent for cell containing the rare nucleic acid to be isolated.

The relative amounts of the various reagents in the kits can be varied widely to provide for concentrations of the reagents that substantially optimize the reactions that need to occur during the present methods and further to optimize substantially the sensitivity of the methods. Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description/instructions of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5. The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted.

### Abbreviations:

K₃EDTA=potassium salt of ethylenediaminetetraacetate
min=minute(s)
μm=micron(s)
mL=milliliter(s)
mg=milligrams(s)
μg=microgram(s)
PBS=phosphate buffered saline (3.2 mM Na₂HPO₄, 0.5 mM KH₂PO₄, 1.3 mM KCl, 135 mM NaCl, pH 7.4)
mBar=millibar
w/w=weight to weight
RT=room temperature
hr=hour(s)
QS=quantity sufficient
Ab=antibody
mAb=monoclonal antibody
vol=volume
MW=molecular weight
wt.=weight
Transfix® tube=10 mL Vacutest Kima blood collection tube containing K₃EDTA and 0.45 mL Transfix®
SKBR cells=SKBR3 human breast cancer cells (ATCC)
WBC=white blood cells
Lysis buffer=5M buffered guanidine thiocyanate, detergent
Capture particle with a specific nucleic acid affinity agent=Magnetic beads with streptavidin bond to a specific nucleic acid affinity agent through a biotin
Specific nucleic acid affinity agent=poly T or CK19 hybridization oligo bound to biotin
Magnetic beads with streptavidin=Microparticles (2.0 mg/mL, 1.5 μm) with streptavidin coating.
Magnetic beads with silica coating=Hydroxyl silica micro particles (1.5 μm)
Porous Matrix=WHATMAN® NUCLEOPORE™ Track Etch matrix, 25 mm diameter and 8.0 and 1.0 μM pore sizes
Wash buffer=Phosphate buffered saline (PBS) with 0.2% TWEEN® 20 surfactant
Elution buffer=25 mM Tris-HCl, pH 8 buffer for non-selective extraction and 25 mM citrate pH 3.1 buffer for selective extraction
Cell affinity agents=cytokeratin 8/18 antibody attached to biotin which specifically binds to SBKR cells.
Proteolytic buffer=25 mM Tris-NaCl, 0.3% proteinase K (Invitrogen CA)
DNase solution=DNase buffer (Qiagen mat#1064143, Qiagen, Inc.) and DNase I (Qiagen mat#1064141, Qiagen, Inc.).

### Example 1

### Selective Cell Free Nucleic Acid Enrichment

The following demonstrates the method of cell free nucleic acid selective enrichment occurring on a nucleic acid binding matrix where nucleic acids are released from nucleic acid binding matrix.

Whole blood specimens were collected from donor or patient (˜8 mL each tube) into Transfix® tubes according to an IRB-approved protocol. Tubes were inverted 20 times and allow them to sit for 24 hours at room temperature (RT). Samples were centrifuged in the Transfix tubes using a swinging bucket at RT, 1700 g, for 20 minutes, and plasma layer on top collected being careful to avoid the buffy coat below it. Plasmas aliquots of 0.5 mL of the plasma were added to 2.5 mL of PBS buffer in polypropylene sterile centrifuge tubes. Nucleic acids were added by counting SKBR human breast cancer cells, lysing the SKBR cells with lysis buffer and adding the cell free nucleic acids to the diluted plasma in form of a cell lysate from 1 to 1000 lysed cells/tube.

For demonstration of selective extraction by a nucleic acid binding matrix, 50 μL of a capture particle with a nucleic acid affinity agent was added to the diluted plasma. As a control 50 μL of magnetic beads with streptavidin was added to the plasma sample. As a second example for 50 μL of magnetic beads with silica coating as a nucleic acid affinity agent was added to the plasma sample. Samples were mixed by inverting, and incubateing the mixture at RT for 15 minutes on a roller mixer at 75 rpm to allow the beads to capture the nucleic acids.

The nucleic acid affinity agent (particles) with bound nucleic acids were isolated by filtration performed by first separation of the particles from the diluted plasma. The cells remaining in the blood pellet were isolated by the standard filtration process such as previously described (Magbanua M J M, Pugia M, Lee J S, Jabon M, Wang V, et al. (2015) A Novel Strategy for Detection and Enumeration of Circulating Rare Cell Populations in Metastatic Cancer Patients Using Automated Microfluidic Filtration and Multiplex Immunoassay. PLoS ONE 10(10)). The only change to the process was to use a vacuum filtration unit (Biotek Inc) and a standard ELISA plate fitted with the standard Whatman membrane with pore holes of 0.8 μm diameter. The sample was filtered through a membrane with pores. During filtration, sample on the porous matrix was subjected to a vacuum of about 100 mBar lower from atmospheric pressure. The nucleic acid affinity agent (particles) captured on the membrane were washed with wash buffer. The nucleic acids were removed from the membrane by washing with elution buffer. To demonstrate that the method allowing both nucleic acid enrichment of both cell free and cellular nucleic acids, both intact and lysed SKBR were added to the same tube. As a comparison of prior art the nucleic acid affinity agent (particles) with bound nucleic acids were isolated by magnetics.

The samples from selective cell free nucleic acid isolation were able to achieve a “minimal purity” of CK and ACTB in the range of 0.01% to about 20% and still achieve the minimal copy number 100 to about 10,000,000 of rare nucleic acid for lysates from 10-50 SBKR cells added to 0.5 mL of whole blood with all the expected nucleic acids. The prior art methods either needed greater purity or did not achieve the minimal copy number.

### Example 2

### Selective Cellular Nucleic Acid Isolation

The following demonstrates the method of cellular nucleic acid selective enrichment occurring on a nucleic acid binding matrix where nucleic acids are released from nucleic acid binding matrix.

Whole blood specimens were collected from donor or patient (˜8 mL each tube) into Transfix® tubes according to an IRB-approved protocol. Tubes were inverted 20 times and allowed to sit for 24 hours at room temperature (RT). Cellular nucleic acids were added by counting SKBR human breast cancer cells, and adding to the blood in form a concentration of 1 to 1000 cells/tube. Whole blood aliquots of 0.5 mL were added to 2.5 mL of PBS buffer in polypropylene sterile centrifuge tubes tube.

For demonstration of selective extraction by a nucleic acid binding matrix, the cells were isolated by a standard filtration process such as previously described (Pugia 2016). As a second example, 50 μL of magnetic beads with streptavidin coated with cell affinity agents which specifically bind SKBR was added to the diluted blood sample prior to filtration. Samples were mixed by inverting, and incubate the mixture at RT for 15 minutes on a roller mixer at 75 rpm to allow the beads to capture the SKBR cells containing cellular nucleic acids. Again, the cells were isolated by a standard filtration process.

Cells were then further reacted with a cell affinity agent, in this case mAb to cytokeratin (CK) that is selectively bound to SBKR cell and not to WBC to allow visualization of minimal purity. In some cases an additional mAb that binds to RNA:DNA is used to selectively bind to additional cellular nucleic acid. In all cases unbound nucleic acid is washed away using a series of liquids following the filtration. In this case the porous matrix was washed with PBS, and the sample was fixed with formaldehyde, washed with PBS, subjected to permeabilization using 0.2% TRITON® X100 in PBS and washed again with PBS. A blocking step was employed in which blocking buffer of 10% casein in PBS was dispensed on the porous matrix prior to adding the cell affinity agents. After an incubation period of 5 min, the matrix was washed with PBS to block non-specific binding to the matrix. Multiple wash buffers were used to wash porous matrix after each affinity reaction. The rare cells were then measured using affinity reactions and immunocytochemistry (ICC) with a fluorescent label attached to the antibody for CK.

The samples from selective cell nucleic acid isolation were able to achieve a “minimal purity” of CK and ACTB in the range of 0.01% to about 20% and still achieve the minimal copy number 100 to about 10,000,000 minimal purity of rare nucleic acid for 10-50 SBKR cells added to 0.5 mL of whole blood with all the expected nucleic acids. The prior art methods either needed greater purity or did not achieve the minimal copy number.

### Example 3

### Selective Nucleic Acid Amplification and Corrected Analysis

The procedure to amplify and analyze nucleic acids isolated was demonstrated with mRNA for CK19 sequence as a disease-related rare nucleic acid and using beta-actin (ACTB) as a reference rare nucleic acids and a reverse-transcription quantitative PCR (RT-qPCR) after the samples of nucleic acid were selectively enriched in Examples 1 or 2. Cell free nucleic acid was demonstrated with samples from Example 1 where nucleic acids isolated on the porous matrix were from the lysed SKBR cells added to blood before filtering. Cellular nucleic acid was demonstrated with samples from Example 2 where nucleic acids isolated on the porous matrix were from the intact SKBR cells added to blood before filtering.

The enriched cell free RNA was removed from the porous matrix by placing the porous matrix in a 1.5 mL tube and the porous matrix was pushed to the bottom of the tube using forceps and combined with 50 μL of lysis buffer containing a protease to release RNA from cells. The tubes were incubated at 55° C. for 60 min with occasional mixing by vortexing. The tubes were then incubated at 65° C. for 15 min with occasional vortexing. The higher temperature was employed to reverse formaldehyde crosslinking of the RNA. The tubes were then incubated at 94° C. for 5 min to deactivate the protease.

The sample was further processed by adding a 10x DNase I buffer (5 μL) and DNase I enzyme to each sample, which were then incubated for 15 min at RT. The solution was removed, and placed in a clean 1.5 mL tube and then processed with the Zymo Quick-RNA MicroPrep kit to clean the RNA from enzymes and elute the RNA into 154, of water. A reverse-transcription quantitative PCR (RT-qPCR) was conducted using the Luna Universal Probe One-step RT-qPCR kit (New England Biolabs, MA). A PCR reaction solution was made by adding forward and reverse primers (0.4 fluorescein (FAM)-labeled probe (0.2 μM) and BSA (1 mg/mL) to the PCR reaction solution and sealing. The selective amplification and corrected detection was conducted on the QuantStudio3 real-time PCR instrument (Applied Biosystems, CA) using Taqman chemistry, standard curve experiment, and cycle threshold analysis of 55° C. for 15 min, 95° C. for 1 min for 1 cycle, and then cycling at 10 sec at 95° C. followed by 60 sec at 60° C. for 1 min for up to 55 cycle, and finally storing the sample at 4° C. Positive and negative controls lacking and containing SKBR lysates were ran.

Only samples from selective nucleic acid isolation from examples 1 and 2 were able to do the CK and ACTB selective amplification and achieve the minimal cycle number while maintaining a high fidelity amplification. The minimal cycle number was always less than 40 amplification cycles for a minimal copy number of CK and ACTB rare nucleic acids. In runs, the minimal cycle number is 10 to about 20 cycles. The method of the inventions was able to detect 10-50 SBKR cells or lysate from 10-50 SBKR cells for whole blood with all the expected nucleic acids contaminations. The DNA minimal copy number for the method was about 100 for whole blood samples. The RNA minimal copy number for the method was about 10,000 for whole blood samples. The correction of CK with ACTB was required to achieve these minimal copy number and minimal cycle number. In corrected nucleic acids analysis, rare nucleic acids analyzed are a combination of disease-related nucleic acids and reference nucleic acids.

The minimal cycle number needed in prior art nucleic acid isolation method was always greater than 40 cycles and it was only able to detect 100-500 SBKR cells or lysate from 100-500 SBKR cells. This was true where the prior art was nucleic acid isolation method using selective or specific nucleic acid affinity agent on particles or spin columns to bind nucleic acids and carry out isolation either by magnetics or centrifugal force. The prior art nucleic acid isolation methods lacked the porous matrix. The lowest number of allowed amplification cycles was >40 cycles for nucleic acids analysis and a high-fidelity amplification was not maintained. While not bound to any mechanism of action, it is believed that the porous matrix allows selective isolation to be in the ideal purity correct range with the ideal minimal copy number to allow selective amplification to perform a corrected nucleic acids analysis.

### Example 4

### Parallel Dual Filtration

Whole blood sample was centrifuged and separated into plasma and cell fractions,

The intact SKBR and cell-free nucleic acids are captured using filtration in parallel. The cell fraction was diluted in buffer and filtered using membrane with 8 μm pores to capture intact SKBR. A SKBR specific antibody coated nanoparticles were used to visualize intact SKBR on membrane by fluorescence microscopy imaging. Particles coated with capturing probes for cell-free DNA/RNA were incubated with the plasma fraction. Particles were then captured and washed using membrane with 1 μM pores. Analysis of intact SKBR and cell-free DNA/RNA: intact SKBR were analyzed using molecular assays; Cell-free DNA/RNA from SKBR was analyzed using molecular assays such as qPCR, ddPCR, and NGS. For molecular assays, cell-free DNA/RNA and SKBR cell lysate were analyzed separately or combined.

### Example 5

### Simultaneous Cell and Cell Free Capturing

Magnetic particles coated with cell-free DNA/RNA probes and SKBR specific antibodies were incubated with whole blood sample. Cell-free DNA/RNA were captured by the probes and intact SKBR bound with the magnetic particles were collected using magnet-based separation and washing. Analysis of intact SKBR and cell-free DNA/RNA was done. Intact SKBR were analyzed by fluorescence microscopy imaging, and molecular assays. Cell-free DNA/RNA were analyzed using molecular assays such as qPCR, ddPCR, and NGS and for molecular assays, cell-free DNA/RNA and SBKR cell lysate were analyzed separately or combined.

### Example 6

### Sequential Cell and Cell Free Filtration

Magnetic particles coated with capturing probes for cell-free DNA/RNA were incubated with whole blood sample. Magnetic particles with captured cell-free DNA/RNA were collected and washed using a magnet. Supernatant from the magnetic separation containing cells and other blood contents. Whole blood sample was diluted in buffer and filtered through a membrane with 8 μm pores to capture intact SBKR. The flow-through from the first filtration was then centrifuged to separate and collect the plasma fraction. The plasma fraction is incubated with particles coated with probes for cell-free DNA/RNA. After incubation, the plasma fraction was filtered through a membrane with smaller pores to capture the particles with captured cell-free DNA/RNA. Analysis of intact SBKR and cell-free DNA/RNA was conducted. Intact SKBR were analyzed using molecular assays. Cell-free DNA/RNA was analyzed using molecular assays such as qPCR, ddPCR, and NGS. For molecular assays, cell-free DNA/RNA and SKBR cell lysate were analyzed separately or combined.

### Example 7

### Sequential Magnetic Separation and Filtration

Magnetic particles coated with capturing probes for cell-free DNA/RNA are incubated with whole blood sample. Magnetic particles with captured cell-free DNA/RNA are collected and washed using a magnet. Supernatant from the magnetic separation containing cells and other blood contents is filtered through a membrane with 8 μm pores to capture intact SKBR. Analysis of intact SKBR and cell-free DNA/RNA was conducted. Intact SKBR was analyzed using fluorescence microscopy imaging, and molecular assays. Cell-free DNA/RNA was analyzed using molecular assays such as qPCR, ddPCR, and NGS. For molecular assays, cell-free DNA/RNA and intact SKBR cell lysate were analyzed separately or combined.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A method for the selective isolation, amplification and detection of nucleic acids from samples, said method comprising:
(a) enriching selectively said nucleic acids present in said samples on a binding matrix;
(b) releasing said nucleic acids from the binding matrix;
(c) selectively amplifying said nucleic acids; and
(d) analysing said amplified nucleic acids.

2. The method according to claim 1, wherein said nucleic acid binding matrix is a porous matrix.

3. The method according to claim 2, wherein said binding matrix optionally includes nucleic acid affinity agents, capture particle, cell affinity agents or hybridization oligos.

4. The method according to claim 2, wherein in said enriched samples the non-rare nucleic acids are removed from the nucleic acid affinity agent by washing solution, and the retained rare nucleic acids are released from the nucleic acid affinity agent using a release solution.

5. The method according to claim 1, wherein the nucleic acids that are released from the nucleic acid binding matrix are selectively amplified from a mixture of disease-related nucleic acids and reference nucleic acids.

6. The method according to claim 1, where said amplified rare nucleic acids are analyzed and corrected by determining the ratio of disease-related nucleic acids to reference nucleic acids to determine whether rare nucleic acid are present.

7. The method according to claim 1, where the amplified rare nucleic acids are measured by quantitative polymerase chain reaction (qPCR) or reverse transcription-qPCR (RT-qPCR).

8. The method according to claim 1, where the selective nucleic acid enrichment generates at least a minimal copy number and higher purity nucleic acids allowing for selective amplification with a minimum number of cycles.

9. The method according to claim 1, wherein said nucleic acids comprise disease-related nucleic acids and reference nucleic acids.

10. The method according to claim 1, wherein said nucleic acids are cellular and cell free, and their enrichment is done separately on a nucleic acid binding matrix.

11. The method according to claim 1, wherein said nucleic acids are cellular and cell free, and their enrichment is done together on a nucleic acid binding matrix.

12. The method according to claim 1, wherein said nucleic acids are cellular and cell free, and said cellular nucleic acids are enriched on a nucleic acid binding matrix and said cell free nucleic acids are not enriched and pass through.

13. The method according to claim 1, wherein said nucleic acids are cellular and cell free, and the cell free nucleic acids are enriched on a nucleic acid binding matrix and said cellular nucleic acids are not enriched and pass through.
